(19) United States
(12) Patent Application Publication
(10) Pub. No.: US 2025/0179157 A1
(43) Pub. Date: Jun. 5, 2025

(54) A-FABP ANTIBODIES FOR DIAGNOSIS AND TREATMENT OF OBESITY-ASSOCIATED DISEASES

(71) Applicant: University of lowa Research Foundation, Iowa City, IA (US)

(72) Inventor: Bing Li, Coralville, IA (US)

(21) Appl. No.: 18/842,152

(22) PCT Filed: Feb. 28, 2023
(86) PCT No.: PCT/US2023/063408

§ 371 Date: Aug. 28, 2024

Related U.S. Application Data
(60) Provisional application No. 63/315,035, filed on Feb. 28, 2022.

(51) Int. Cl. C07K 16/18; A61K 39/00; A61P 3/10; A61P 35/00

(52) U.S. Cl. CPC C07K 16/18; A61P 3/10; A61P 35/00; A61K 2039/505; C07K 2317/24; C07K 2317/76

(57) ABSTRACT

An anti-human A-FABP antibody, or an antigen binding fragment thereof, or a polypeptide, that inhibits human A-FABP activity, and uses thereof, are provided.

### CROSS-REFERENCE TO RELATED APPLICATIONS

This application claims the benefit of the filing date of U.S. application No. 63/315,035, filed on Feb. 28, 2022, the disclosure of which is incorporated by reference herein.

### INCORPORATION BY REFERENCE OF SEQUENCE LISTING

A Sequence Listing is provided herewith as an xml file, “2313249.xml” created on Feb. 27, 2023, and having a size of 191,264 bytes. The content of the xml file is incorporated by reference herein in its entirety.

### BACKGROUND

While multiple factors, including aging, genetic background, family and reproductive history, contribute to breast cancer (BC) risk, epidemiologic studies have confirmed that obesity increases the risk of BC in postmenopausal women and the mortality from BC in women of all ages. Due to excess intake of dietary calories and a sedentary lifestyle in modern society, obesity has risen at an alarming rate in the US (adult obesity prevalence reached to 42.4% in 2017-2018 according to CDC statistics). As a result, extra energy stored as lipids in various cells and tissues can negatively affect cell metabolism and homeostasis. Compared to adipose tissue in lean individuals, adipose tissue in the obese patients contains crown-like structures formed by hypertrophic adipocytes surrounded by macrophages. The interaction between macrophages and adipocytes promotes obesity-associated chronic inflammation and further pathological alterations. As an endocrine organ, adipose tissue produces multiple mediators (e.g. adipokines, cytokines, chemokines and hormones) to maintain metabolic balance. Dysregulation of these mediators leads to obesity-associated diseases, including BC. Given the prevalence of obesity and increased risk of BC in obese patients, there is an urgent need to identify obesity-associated metabolic targets in BC risk.

Overconsumption of HFDs has been linked to the obesity epidemic. Dietary fatty acids (FAs) are either saturated, such as 16:0 palmitic acid (PA) and 18:0 stearic acid (SA), or unsaturated, such as 18:1 monounsaturated oleic acid (OA) and 18:2 polyunsaturated linoleic acid (LA). Polyunsaturated FAs include omega-6 (e.g. LA) and omega-3 (e.g. docosapentaenoic acid, DPA; eicosapentaenoic acid, EPA) FAs, depending on the double bond position counting from the methyl end of the carbon chain. Different HFDs have distinct FA compositions. For example, cocoa butter HFD is rich in saturated FAs, but fish oil HFD is rich in omega-3 FAs. It was demonstrated that while both cocoa butter and fish oil HFDs can induce similar levels of obesity, only cocoa butter-induced obesity is associated with increased A-FABP levels and accelerated mammary tumor growth. Given the different FA components in HFDs, it is of interest to determine whether and how FA composition in HFDs influences obesity and obesity-associated lipid dysregulation and BC risk. Due to its characteristic expression profile in macrophages and adipocytes, A-FABP has been studied in obesity-related metabolic diseases, including type 2 diabetes, atherosclerosis and heart diseases.

Fatty acids are essential energy to health. However, dietary fatty acids are insoluble in aqueous environment. Fatty acid binding proteins (FABPs) are evolutionarily conserved proteins which bind and transport fatty acids to facility fatty acid-mediated responses. The family of FABPs consists of at least 9 members which exhibit unique expression pattern of tissue distribution. In the adipose tissue, adipose FABP (A-FABP, also known as FABP4, ap2) is highly expressed in adipocytesand macrophages, facilitating fatty acid storage and export. A-FABP is usually expressed inside cells, but during obesity, A-FABP can be released into the circulation.

### SUMMARY

Elevated circulating A-FABP promotes at least some obesity-associated diseases, including, but not limited to, breast cancer, type 2 diabetes, or atherosclerosis. As described herein, inhibition of A-FABP activity may provide for treatment of, in one embodiment, obesity-associated diseases and measuring circulating levels of A-FABP may provide for diagnosis of, for example, the severity of obesity-associated diseases. In one embodiment, anti-A-FABP antibodies, e.g., humanized anti-A-FABP antibodies, may be employed for A-FABP measurement and/or prevention, inhibition or treatment of A-FABP-mediated diseases.

A-FABP antibodies were secreted from a hybridoma, which was obtained by immunizing mice with human A-FABP. After in vitro screening of multiple clones of anti-A-FABP hybridomas, positive clones were selected for both in vitro and in vivo functional testing. Clones were sequenced to identify the unique antibody variable regions, including the complementarity determining regions (CDR) regions. Then CDR regions were grafted to human IgG antibodies with back mutations to create multiple humanized anti-A-FABPs with unique antigen epitopes.

The disclosure provides a composition comprising a humanized anti-human A-FABP antibody, or an antigen binding fragment thereof, or a polypeptide, that inhibits human A-FABP activity, wherein the antibody, the antigen binding fragment thereof, or the polypeptide has: i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6). In one embodiment, the antibody fragment is a scFv. In one embodiment, the antibody comprises an IgG1, IgG2, IgG4, IgM, IgA, or IgE heavy chain constant region. In one embodiment, the antibody comprises an Ig kappa light chain constant region. In one embodiment, the antibody comprises an Ig lambda light chain constant region. In one embodiment, the composition further comprises a pharmaceutically acceptable carrier. In one embodiment, the antibody, fragment thereof, or polypeptide comprises one of SEQ ID Nos. 7 to 38. In one embodiment, the antibody, fragment thereof, or polypeptide comprises a region having at least 80% amino acid sequence identity to the non-CDR regions in one of SEQ ID Nos. 7 to 38.

In one embodiment, the CDR has GFNIKNTY (SEQ ID NO:1), IDPANGNT (SEQ ID NO:2), VSLTGVFAY (SEQ ID NO:3), or QHFWGTPWT (SEQ ID NO:6) or a sequence with 1, 2, 3, 4, or 5 substitutions, e.g., 1 or more conservative substitutions, or the CDR has ENIYSN (SEQ ID NO:4) or AAT, or a sequence with 1 or 2 substitutions, e.g., 1 or more conservative substitutions.

Also provided is an isolated cell comprising an expression cassette comprising a heterologous promoter operably linked to nucleic acid sequences encoding a humanized anti-human A-FABP antibody, or an antigen binding fragment thereof, or a polypeptide, that inhibits human A-FABP activity, wherein the antibody, the antigen binding fragment thereof, or the polypeptide has: i) a variable region comprising a first complementarity determining region (CDR) comprising (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6). In one embodiment, the cell is a mammalian cell, e.g., a Chinese hamster ovary (CHO) cell. In one embodiment, the cell is a primate cell. In one embodiment, the cell is a human cell.

In one embodiment, an isolated nucleic acid is provided comprising a promoter operably linked to a nucleotide sequence which encodes at least the variable region of a human heavy or light chain that binds human A-FABP, wherein the chain comprises: i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6). In one embodiment, the promoter is a heterologous promoter.

Also provided is a method to prevent, inhibit or treat obesity-associated cancers, including 13 types of cancers known to be significantly associated with obesity, for example, breast cancer, leukemia, pancreatic cancer, multiple myeloma, colorectal cancer, kidney cancer, endometrial cancer, thyroid cancer, liver cancer, ovarian cancer, adenocarcinoma of the esophagus, gastric cardia cancer, gallbladder cancer, or meningioma, in a mammal, comprising: administering to a mammal a composition comprising an effective amount of an antibody or fragment thereof comprising, a polypeptide comprising or nucleotide sequence which encodes, at least the variable region of a human heavy or light chain that binds human A-FABP, wherein the chain comprises: i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6), or an antibody or fragment comprising i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6). In one embodiment, the heavy chain is an IgG heavy chain. In one embodiment, the light chain is an Ig K light chain. In one embodiment, the antibody fragment is administered. In one embodiment, the fragment is Fab′ or scFv.

A method to diagnose the risk of obesity-associated cancers, including breast cancer, by measuring body fluid levels of A-FABP in a mammal is provided comprising: a) contacting a physiological sample from the mammal and at least the variable region of a human heavy or light chain that binds human A-FABP, wherein the chain comprises: i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6), or an antibody or fragment comprising i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6); and b) determining the presence or amount of A-FABP in the sample. In one embodiment, the sample is a physiological fluid sample. In one embodiment, the sample is a blood sample. In one embodiment, the sample is a plasma sample. In one embodiment, the mammal is human.

Further provided is a method to prevent, inhibit or treat other obesity-associated diseases, including type 2 diabetes, atherosclerosis, heart disease or stoke in a mammal. The method includes administering to a mammal a composition comprising an effective amount of an antibody or fragment thereof comprising, a polypeptide comprising or nucleotide sequence which encodes, at least the variable region of a human heavy or light chain that binds human A-FABP, wherein the chain comprises: i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6), or an antibody or fragment comprising i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or ii) a variable region comprising a first CDR comprising ENIYSN (SEQ ID NO:4) operably linked to a second CDR comprising AAT operably linked to a third CDR comprising QHFWGTPWT (SEQ ID NO:6). In one embodiment, the heavy chain is an IgG heavy chain. In one embodiment, the light chain is an Igκ light chain. In one embodiment, the antibody fragment is administered. In one embodiment, the fragment is Fab′ or scFv. In one embodiment, the mammal is human. In one embodiment, the mammal has heart disease. In one embodiment, the mammal has type 2 diabetes. In one embodiment, the mammal has atherosclerosis. In one embodiment, the mammal is obese.

### BRIEF DESCRIPTION OF THE FIGURES

FIGS. 1A-ID. A-FABP upregulation in human BC. A, PROGgene analysis of A-FABP expression with overall survival of BC patients using the TCGA dataset. B, analysis of A-FABP expression in different subtypes of BC using the GEO dataset GSE9014. C, D, H&E staining and confocal analysis of A-FABP expression in normal and malignant human breast tissues. Average A-FABP+ cells in high-power fields (400×) of normal (n=12) and malignant (n=23) human breast tissues are shown in panel D (****p<0.0001, ***p<0.001; **p<0.01; *p<0.05).

FIGS. 2A-2F. A-FABP deficiency in mice reduces tumor growth and metastasis. A, B,
E0771 mammary tumor cells (1×10⁶) were orthotopically injected in the mammary pad of WT and A-FABP^{-/-} mice (n=9/group). Tumor growth (A) and lung metastasis on day 26 after tumor implantation (B) were measured. C, D, MMT060562 cells (1×10⁶) were orthotopically injected into WT and A-FABP^{-/-} mice (n=9/group). Tumor size (C) and lung metastasis (D) were measured similarly as E0771 cells. E, F, MC38 colon tumor cells (1×10⁶) were s.c. injected in the flanks of WT and A-FABP^{-/-} mice (n=9/group). MC38 tumor
growth and lung metastasis are shown in panel E and F (*p<0.05, **p<0.01).

FIGS. 3A-3C. Increased levels of circulating A-FABP in obesity are associated with BC risk. A, correlation analysis between serum A-FABP levels and BMI (n=84). B,C, serum levels of A-FABP in obese (BMI>30) and nonobese (BMI≤30) patients without (B) or with (C) BC(*p<0.05, ****p<0.0001).

FIGS. 4A-4B. Treatment with anti-A-FABP mAb 12G2 inhibits mammary/breast tumor growth in vivo. A, 0.5×10⁶ E0771 cells were orthotopically implanted in C57B/6 mice and treated with PBS control, mouse 6H10, or mouse 12G2 mAb, respectively (n=9/group), from day 3 post tumor implantation (twice/week for 2 weeks at a dose of 30 mg/kg). Tumor growth was measured. B, 3×10⁶ human MCF-7 cells were orthotopically implanted in SCID mice and treated with PBS, chimeric 6H10, or 12G2 mAb, respectively (n=9/group), from day 3 post tumor implantation (twice/week for 3 weeks at a dose of 30 mg/kg). Tumor growth was measured (***p<0.001).

FIG. 5. Inhibition of inflammatory IL-6 cytokine production in diabetic mice treated with different anti-A-FABP antibodies. 8-week-old female diabetic DB/DB mice (n=6) were treated with different anti-A-FABP antibodies twice/week (30 mg/kg) for 2 weeks. Mouse serum was collected for measurement of cytokine levels using Millipore cytokine assay kit (*p<0.05).

FIGS. 6A-6C. Development of humanized anti-A-FABP antibodies. A, B, the CDR regions of chimeric 12G2 antibody were grafted to the closest matching human germline human IgG1 antibodies with back mutations to create 16 humanized anti-A-FABP antibodies (B). C, analysis of humanized anti-A-FABP antibody binding affinity with ELISA.

FIGS. 7A-7B. Humanized anti-A-FAB antibodies inhibit A-FABP-mediated BC colony formation and proliferation in vitro. A, A-FABP-mediated colony formation of breast cancer MCF-7 cells was inhibited by treatment with different humanized anti-A-FABP antibodies. B, Migration of breast cancer MCF-7 cells in response to A-FABP treatment (200 ng/ml) for 96 h was inhibited by treatment with different variants of humanized anti-A-FABP antibodies. (*p<0.05, **p<0.01, ***p<0.001).

FIGS. 8A-8C. Treatment with humanized anti-A-FABP antibodies inhibited mammary tumor growth in vivo. A, mice (n=5/group) were orthotopically implanted E0771 cells (0.5×106) cells were treated with or without selected humanized anti-A-FABP antibody for two weeks (30 mg/kg, twice/week). Tumor growth curve was measured. Tumor pictures and weight are shown in panel B, and C, respectively (**p<0.05 as compared to the WT control group).

FIGS. 9A-9D. Humanized anti-A-FABP treatment inhibited IL-6 production in tumor-infiltrating macrophages and ALDH1 activity in tumor cells. A, B, measurement of intracellular IL-6 production in tumor infiltrating macrophages in mice treated with or without a humanized anti-A-FABP antibody. Average percentage of IL-6⁺ macrophages is shown in panel B. C, D, analysis of cancer sternness marker ALDH1 activity in tumor cells by flow cytometric staining. Average percentage of ALDH1⁺ tumor cells is shown in panel D (n=5/group, **p<0.05).

FIGS. 10A-10B. Treatment with humanized anti-A-FABP antibodies inhibited breast cancer growth in vivo. SCID were fed either normal chow diet (A) or high fat diet (B) for 3 months. Lean SCID mice on chow diet (A) or obese SCID mice on the high fat diet (B) (n=10/group) were orthotopically implanted human MCF-7 cells (8×10⁸/mouse) were treated with or without selected humanized anti-A-FABP antibody for 4 weeks (10 mg/kg, twice/week). Tumor volume was measured in control or antibody treated group, respectively (p<0.0001 as compared to the control group).

FIGS. 11A-11B. Humanized anti-A-FABP antibody treatment reduces glucose levels in ob/ob mice. A, resting blood glucose levels in ob/ob mice s.c. treated with PBS or mAbs (5 mg/kg) twice a week for 4 weeks. B, glucose tolerance test (GTT) was performed in ob/ob mice after treatment with PBS or humanized mAbs for 4 weeks. Glucose was injected at 2 g/kg after 15 hours of fasting (n=4/group).

FIG. 12. Variable and heavy region sequences (VH having SEQ ID Nos. 179-194 and VL having SEQ ID Nos. 195-210).

### DETAILED DESCRIPTION

Fats are essential nutrients in human diets as they function as cellular building blocks, an efficient source of energy and signaling molecules regulating intracellular and extracellular activities. Given that fats are insoluble in the aqueous environment of living organisms, fatty acid binding proteins (FABPs) are identified as lipid chaperones, playing a central role in coordinating lipid transport, metabolism and responses in various tissues and organs. The family of FABPs consists of at least nine members, each exhibiting distinct pattern of tissue distribution. For example, adipose FABP (A-FABP, also known as FABP4) is mainly expressed in adipose tissues, including adipocytes and macrophages. It has been observed that host expression of A-FABP promotes obesity-associated breast cancer (BC) risk through at least two aspects: 1) Enhancing pro-tumor functions in tumor associated macrophages (TAMs). A-FABP is intracellular expressed in a specific subset of TAMs promoting inflammatory cytokine IL-6 production and oncogenic STAT3 signaling, and 2) Increasing BC cell aggressive phenotypes. Obesity elevates A-FABP secretion from adipose tissue into the circulation. Thus, A-FABP might represent a new factor linking dysregulated lipid metabolism to obesity-associated cancer risk, including BC. Moreover, it was observed that obesity can be induced by consumption of different types of HFDs, including saturated fats (e.g. cocoa butter) and unsaturated fats (e.g. olive oil, fish oil). However, only cocoa butter HFD-induced obesity was associated with increased A-FABP expression and mammary tumor growth. These observations suggest that not all HFD-induced obesity is tumorigenic. Given the undefined links underlying obesity-associated BC risk, it was hypothesized that HFDs rich in saturated fats promote BC risk through an A-FABP-mediated immune and metabolic regulations. As such, A-FABP offers a therapeutic target and biomarker for obesity-associated cancer risk.

### Definitions

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the disclosure described herein that is a polynucleotide encompasses both the double-stranded form and each of two complementary single-stranded forms known or predicted to make up the double-stranded form.

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this disclosure are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present disclosure generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. A typical example of such sequence-specific terminators includes polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present disclosure are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, e.g., 6 bases or less or 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

The term “antibody,” as used herein, may refer to a full-length immunoglobulin molecule or an immunologically-active fragment of an immunoglobulin molecule such as the Fab or F(ab′)2 fragment generated by, for example, cleavage of the antibody with an enzyme such as pepsin or co-expression of an antibody light chain and an antibody heavy chain in, for example, a mammalian cell, or ScFv. The antibody can also be an IgG, IgD, IgA, IgE or IgM antibody. Full-length immunoglobulin “light chains” (about 25 kD or 214 amino acids) are encoded by a variable region gene at the amino-terminus (about 110 amino acids) and a kappa or lambda constant region gene at the carboxy-terminus. Full-length immunoglobulin “heavy chains” (about 50 kD or 446 amino acids), are similarly encoded by a variable region gene (about 116 amino acids) and one of the other aforementioned constant region genes, e.g., gamma (encoding about 330 amino acids). Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (VH) refer to these light and heavy chains respectively. In each pair of the tetramer, the light and heavy chain variable regions are together responsible for binding to an antigen, and the constant regions are responsible for the antibody effector functions. In addition to naturally occurring antibodies, immunoglobulins may exist in a variety of other forms including, for example, Fv, ScFv, Fab, and F(ab′)₂, as well as bifunctional hybrid antibodies (e.g., Lanzavecchia et al. (1987)) and in single chains (e.g., Huston et al. (1988) and Bird et al. (1988), which are incorporated herein by reference). (See, generally, Hood et al., “Immunology”, Benjamin, N.Y., 2^{nd} ed. (1984), and Hunkapiller and Hood (1986), which are incorporated herein by reference). Thus, the term “antibody” includes antigen binding antibody fragments, as are known in the art, including Fab, Fab₂, single chain antibodies (scFv for example), chimeric antibodies, etc., either produced by the modification of whole antibodies or those synthesized de novo using recombinant DNA technologies.

An immunoglobulin light or heavy chain variable region consists of a “framework” region interrupted by three hypervariable regions, also called CDR's. The extent of the framework region and CDR's have been precisely defined (see, “Sequences of Proteins of Immunological Interest,” E. Kabat et al., U.S. Department of Health and Human Services, (1983); which is incorporated herein by reference). The sequences of the framework regions of different light or heavy chains are relatively conserved within a species. As used herein, a “human framework region” is a framework region that is substantially identical (about 85% or more, usually 90 to 95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. One example of a chimeric antibody is one composed of the variable or antigen-binding domain from a mouse antibody and the constant or effector domain from a human antibody, although other mammalian species may be used.

As used herein, the term “humanized” immunoglobulin refers to an immunoglobulin having a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they are generally substantially identical to human immunoglobulin constant regions, i.e., at least about 85-90%, or about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin. One says that the donor antibody has been “humanized”, by the process of “humanization”, because the resultant humanized antibody is expected to bind to the same antigen as the donor antibody that provides the CDR's.

Thus, humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody has substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will include at least a portion of an immunoglobulin constant region (Fc), typically that of a human immunoglobulin (Jones et al. (1986); Riechmann et al. (1988); and Presta (1992)).

It is understood that the humanized antibodies may have additional conservative amino acid substitutions which have substantially no effect on antigen binding or other immunoglobulin functions. By conservative substitutions are intended combinations such as gly, ala; val, ile, leu; asp, glu; asn, gln; ser, thr; lys, arg; and phe, tyr.

Humanized immunoglobulins, including humanized antibodies, have been constructed by means of genetic engineering. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522 (1986); Riechmann et al., Nature, 332:323 (1988); Verhoeyen et al., Science, 239:1534 (1988)), by substituting rodent CDRs or CDR sequences for the corresponding sequences of a human antibody. Accordingly, such “humanized” antibodies are chimeric antibodies that have substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147:86 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779 (1992); Lonberg et al., Nature, 368:856 (1994); Morrison, Nature, 368:812 (1994); Fishwild et al., Nature Biotechnology, 14:845 (1996); Neuberger, Nature Biotechnology, 14:826 (1996); Lonberg and Huszar, Intern. Rev. Immunol., 13:65 (1995). Most humanized immunoglobulins that have been previously described have a framework that is identical to the framework of a particular human immunoglobulin chain and three CDR's from a non-human donor immunoglobulin chain.

A framework may be one from a particular human immunoglobulin that is unusually homologous to the donor immunoglobulin to be humanized, or a consensus framework derived from many human antibodies. For example, comparison of the sequence of a mouse heavy (or light) chain variable region against human heavy (or light) variable regions in a data bank (for example, the National Biomedical Research Foundation Protein Identification Resource) shows that the extent of homology to different human regions varies greatly, typically from about 40% to about 60-70%. By choosing one of the human heavy (respectively light) chain variable regions that is most homologous to the heavy (respectively light) chain variable region of the other immunoglobulin, fewer amino acids will be changed in going from the one immunoglobulin to the humanized immunoglobulin. The precise overall shape of a humanized antibody having the humanized immunoglobulin chain may more closely resemble the shape of the donor antibody, also reducing the chance of distorting the CDR's.

Typically, one of the 3-5 most homologous heavy chain variable region sequences in a representative collection of at least about 10 to 20 distinct human heavy chains is chosen as acceptor to provide the heavy chain framework, and similarly for the light chain. One of the 1 to 3 most homologous variable regions may be used. The selected acceptor immunoglobulin chain may have at least about 65% homology in the framework region to the donor immunoglobulin.

In many cases, it may be considered desirable to use light and heavy chains from the same human antibody as acceptor sequences, to be sure the humanized light and heavy chains will make favorable contacts with each other. Regardless of how the acceptor immunoglobulin is chosen, higher affinity may be achieved by selecting a small number of amino acids in the framework of the humanized immunoglobulin chain to be the same as the amino acids at those positions in the donor rather than in the acceptor.

Humanized antibodies generally have advantages over mouse or in some cases chimeric antibodies for use in human therapy: because the effector portion is human, it may interact better with the other parts of the human immune system (e.g., destroy the target cells more efficiently by complement-dependent cytotoxicity (CDC) or antibody-dependent cellular cytotoxicity (ADCC)); the human immune system should not recognize the framework or constant region of the humanized antibody as foreign, and therefore the antibody response against such an antibody should be less than against a totally foreign mouse antibody or a partially foreign chimeric antibody.

DNA segments having immunoglobulin sequences typically further include an expression control DNA sequence operably linked to the humanized immunoglobulin coding sequences, including naturally-associated or heterologous promoter regions. Generally, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other immunoglobulin forms may follow (see, S. Beychok, Cells of Immunoglobulin Synthesis, Academic Press, New York, (1979), which is incorporated herein by reference).

Other “substantially homologous” modified immunoglobulins to the native sequences can be readily designed and manufactured utilizing various recombinant DNA techniques well known to those skilled in the art. For example, the framework regions can vary at the primary structure level by several amino acid substitutions, terminal and intermediate additions and deletions, and the like. Moreover, a variety of different human framework regions may be used singly or in combination as a basis for the humanized immunoglobulins of the present disclosure. In general, modifications of the genes may be readily accomplished by a variety of well-known techniques, such as site-directed mutagenesis (see, Gillman and Smith, Gene, 8:81 (1979) and Roberts et al., Nature, 328:731 (1987), both of which are incorporated herein by reference). Substantially homologous immunoglobulin sequences are those which exhibit at least about 85% homology, usually at least about 90%, or at least about 95% homology with a reference immunoglobulin protein.

Alternatively, polypeptide fragments comprising only a portion of the primary antibody structure may be produced, which fragments possess one or more immunoglobulin activities (e.g., antigen binding). These polypeptide fragments may be produced by proteolytic cleavage of intact antibodies by methods well known in the art, or by inserting stop codons at the desired locations in vectors known to those skilled in the art, using site-directed mutagenesis.

### Exemplary Gene Transfer Vectors

The disclosure also provides a gene transfer vector comprising a nucleic acid sequence which encodes an antibody, an antigen binding fragment thereof, or a polypeptide, directed against A-FABP. In one embodiment, the gene transfer vector is a virus. The disclosure further provides a method of using the gene transfer vector or encoded gene product against A-FABP in a mammal, which method comprises administering to the mammal the above-described gene transfer vector or the encoded gene product. Various aspects of the gene transfer vector, antibody or antigen binding fragment thereof, and methods are discussed below. Although each parameter is discussed separately, the gene transfer vector, antibody or antigen binding fragment thereof, or polypeptide, and method, may comprise combinations of the parameters set forth below. Accordingly, any combination of parameters can be used according to the gene transfer vector, antibody or antigen binding fragment thereof, the polypeptide, and the method.

A “gene transfer vector” is any molecule or composition that has the ability to carry and deliver a heterologous nucleic acid sequence into a suitable host cell where synthesis of the encoded protein takes place. Typically, a gene transfer vector is a nucleic acid molecule that has been engineered, using recombinant DNA techniques that are known in the art, to incorporate the heterologous nucleic acid sequence. Desirably, the gene transfer vector is comprised of DNA. Examples of suitable DNA-based gene transfer vectors include plasmids and viral vectors. However, gene transfer vectors that are not based on nucleic acids, such as liposomes, are also known and used in the art. The gene transfer vector can be based on a single type of nucleic acid (e.g., a plasmid) or non-nucleic acid molecule (e.g., a lipid or a polymer). The gene transfer vector can be integrated into the host cell genome, or can be present in the host cell in the form of an episome.

In one embodiment, the gene transfer vector is a viral vector. Suitable viral vectors include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. These viral vectors can be prepared using standard recombinant DNA techniques described in, for example, Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994).

Any viral vector may be employed to deliver antibody encoding sequences to cells including mammalian cells, or to mammals, include but are not limited to adeno-associated virus, adenovirus, herpesvirus, retrovirus, or lentivirus vectors.

In addition to the nucleic acid sequence encoding an antibody against A-FABP, or an antigen-binding fragment thereof, the viral vector may comprise expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, CA. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27:4324 (1999); Nuc. Acid. Res., 28:e99 (2000); U.S. Pat. No. 7,112,715; and Kramer & Fussenegger, Methods Mol. Biol., 308:123 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. In one embodiment, the nucleic acid sequence encoding an antibody against A-FABP, or an antigen-binding fragment thereof, is operably linked to a CMV enhancer/chicken beta-actin promoter (also referred to as a “CAG promoter”) (see, e.g., Niwa et al., Gene, 108:193 (1991); Daly et al., Proc. Natl. Acad. Sci. U.S.A., 96:2296 (1999); and Sondhi et al., Mol. Ther., 15:481 (2007)).

Typically AAV vectors are produced using well characterized plasmids. For example, human embryonic kidney 293T cells are transfected with one of the transgene specific plasmids and another plasmid containing the adenovirus helper and AAV rep and cap genes (specific to AAVrh.10, 8 or 9 as required). After 72 hours, the cells are harvested and the vector is released from the cells by five freeze/thaw cycles. Subsequent centrifugation and benzonase treatment remove cellular debris and unencapsidated DNA. Iodixanol gradients and ion exchange columns may be used to further purify each AAV vector. Next, the purified vector is concentrated by a size exclusion centrifuge spin column to the required concentration. Finally, the buffer is exchanged to create the final vector products formulated (for example) in 1× phosphate buffered saline. The viral titers may be measured by TaqMan® real-time PCR and the viral purity may be assessed by SDS-PAGE.

### Exemplary Pharmaceutical Compositions and Delivery

The disclosure provides a composition comprising, consisting essentially of, or consisting of the above-described antibody, antibody fragment, such as a single chain polypeptide, polypeptide, or gene transfer vector and a pharmaceutically acceptable (e.g., physiologically acceptable) carrier, or an antibody or antigen binding fragment, polypeptide, or gene transfer vector thereof optionally with a pharmaceutically acceptable (e.g., physiologically acceptable) carrier. When the composition consists essentially of the antibody, antibody fragment, e.g., single chain polypeptide, polypeptide, or gene transfer vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the gene transfer vector and the pharmaceutically acceptable carrier, or the antibody, antigen binding fragment thereof or polypeptide optionally with a pharmaceutically acceptable carrier, the composition does not comprise any additional components. Any suitable carrier can be used within the context of the disclosure, and such carriers are well known in the art. The choice of carrier will be determined, in part, by the particular site to which the composition may be administered and the particular method used to administer the composition. The composition optionally can be sterile with the exception of the gene transfer vector or an antibody or antigen binding fragment thereof or polypeptide described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene transfer vector, antibody or antigen binding fragment thereof or polypeptide is administered in a composition formulated to protect the gene transfer vector or antibody or antigen binding fragment thereof or polypeptide from damage prior to administration. For example, the composition can be formulated to reduce loss of the gene transfer vector, antibody or fragment thereof or polypeptide on devices used to prepare, store, or administer the gene transfer vector, such as glassware, syringes, or needles. The composition can be formulated to decrease the light sensitivity and/or temperature sensitivity of the gene transfer vector or an antibody or antigen binding fragment thereof. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the gene transfer vector, facilitate administration, and increase the efficiency of the method. Formulations for gene transfer vector-containing compositions are further described in, for example, Wright et al., Curr. Opin. Drug Discov. Devel., 6(2): 174-178 (2003) and Wright et al., Molecular Therapy, 12: 171-178 (2005))

The composition also can be formulated to enhance transduction efficiency. In addition, one of ordinary skill in the art will appreciate that the gene transfer vector or antibody or antigen binding fragment thereof or polypeptide can be present in a composition with other therapeutic or biologically-active agents. For example, factors that control inflammation, such as ibuprofen or steroids, can be part of the composition to reduce swelling and inflammation associated with in vivo administration of the gene transfer vector or the antibody or antigen binding fragment thereof or polypeptide. Immune system stimulators or adjuvants, e.g., interleukins, lipopolysaccharide, and double-stranded RNA, can be administered to enhance or modify the anti-A-FABP immune response. Antibiotics, i.e., microbicides and fungicides, can be present to treat existing infection and/or reduce the risk of future infection, such as infection associated with gene transfer procedures.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, a formulation of the present disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the gene transfer vector, antibody or antigen binding fragment thereof. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Delivery of the compositions comprising the gene transfer vectors, antibody or antigen binding fragment thereof or polypeptide, may be intracerebral (including but not limited to intraparenchymal, intraventricular, or intracisternal), intrathecal (including but not limited to lumbar or cisterna magna), or systemic, including but not limited to intravenous, oral, or any combination thereof, using devices known in the art. Delivery may also be via surgical implantation of an implanted device.

The dose of the active agent in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the gene transfer vector, antibody or antigen binding fragment thereof or polypeptide described herein. A “therapeutically effective amount” refers to an amount effective, at dosages and for periods of time necessary, to achieve a desired therapeutic result. The therapeutically effective amount may vary according to factors such as the extent of pathology, age, sex, and weight of the individual, and the ability of the gene transfer vector, antibody or antigen binding fragment thereof to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. A therapeutically effective amount may be between 1×10¹⁰ genome copies to 1×10¹³ genome copies. A therapeutically effective amount may be between 1×10¹² genome copies to 1×10¹⁵ genome copies (total). A therapeutically effective amount may be between 1×10¹² genome copies/kg to 1×10¹⁵ genome copies/kg.

The dose of antibody or antigen binding fragment thereof or polypeptide in the composition required to achieve a particular therapeutic effect typically is administered in units of antibody or antigen binding fragment or polypeptide per kg (mg/kg) or total dose (mg). One of ordinary skill in the art can readily determine an appropriate dose range to treat a patient having a particular disease or disorder, based on these and other factors that are well known in the art. A therapeutically effective amount of antibody or antigen binding fragment or polypeptide thereof may be between 25 to 200 mg, e.g., 50 to 100 mg, 25 to 50 mg, 50 to 75 mg, 100 to 150 mg, 150 to 200 mg, 200 mg to 300 mg, 300 mg to 400 mg, 400 mg to 500 mg, or 500 mg to 600 mg. A therapeutically effective amount of antibody or antigen binding fragment thereof or polypeptide may be between 1 mg/kg to 20 mg/kg, e.g., 2 to 5 mg/kg, 5 to 7 mg/kg or 10 to 15 mg/kg.

In one embodiment, the composition is administered once to the mammal. It is believed that a single administration of the composition will result in persistent expression of the anti-A-FABP antibody or fragment in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence which encodes an antibody directed against A-FABP, or a therapeutically effective amount of the antibody or antigen binding fragment thereof or polypeptide as described above.

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals are envisioned as subjects, such as non-human primates, sheep, dogs, cats, cows and horses. The subject may also be livestock such as, cattle, swine, sheep, poultry, and horses, or pets, such as dogs and cats.

Exemplary subjects include human subjects suffering from or at risk for the medical diseases and conditions described herein. The subject is generally diagnosed with the condition of the subject disclosure by skilled artisans, such as a medical practitioner.

The methods of the disclosure described herein can be employed for subjects of any species, gender, age, ethnic population, or genotype. Accordingly, the term subject includes males and females, and it includes elderly, elderly-to-adult transition age subject adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the disclosure may be more appropriate for some ethnic populations such as Caucasians, especially northern European populations, as well as Asian populations.

The term subject also includes subjects of any genotype or phenotype as long as they are in need of the disclosure, as described above. In addition, the subject can have the genotype or phenotype for any hair color, eye color, skin color or any combination thereof. The term subject includes a subject of any body height, body weight, or any organ or body part size or shape.

The invention will be further described by the following non-limiting examples.

### Example 1

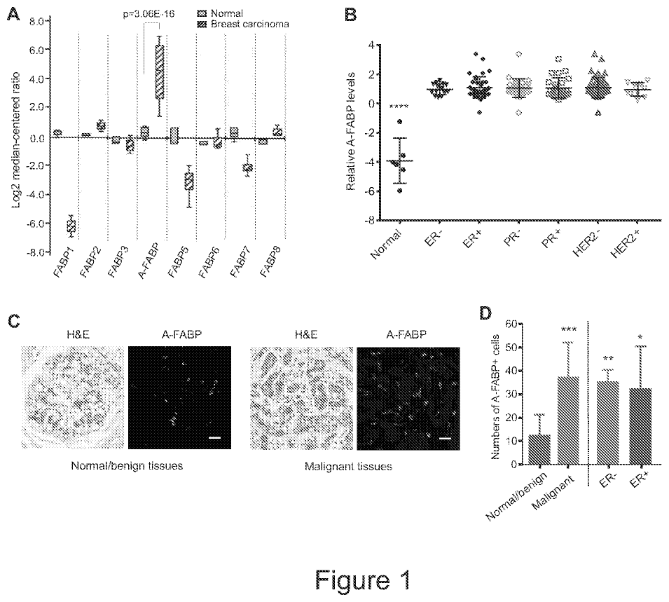

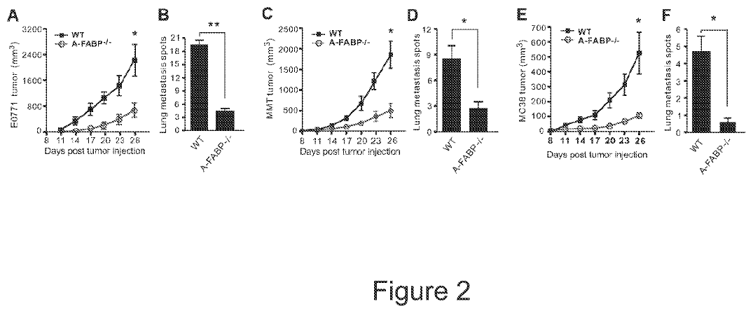

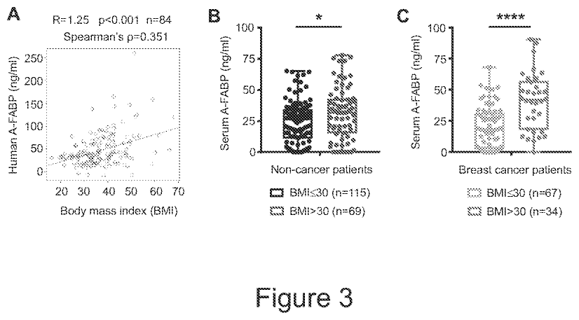

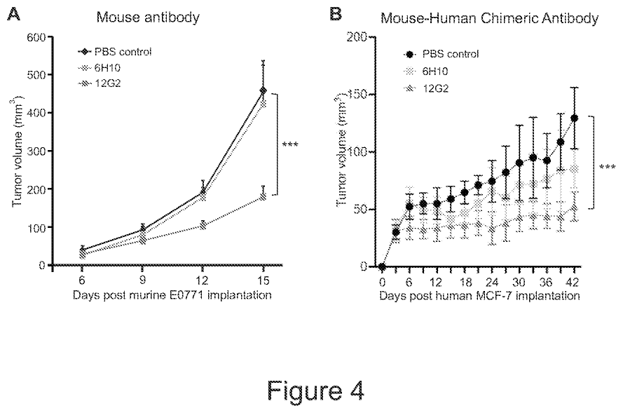

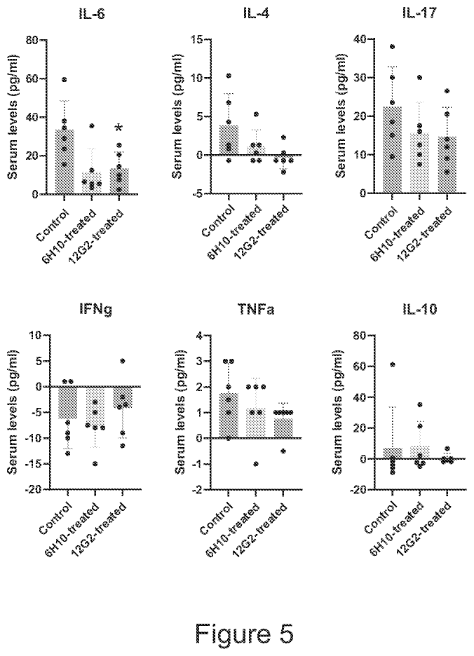

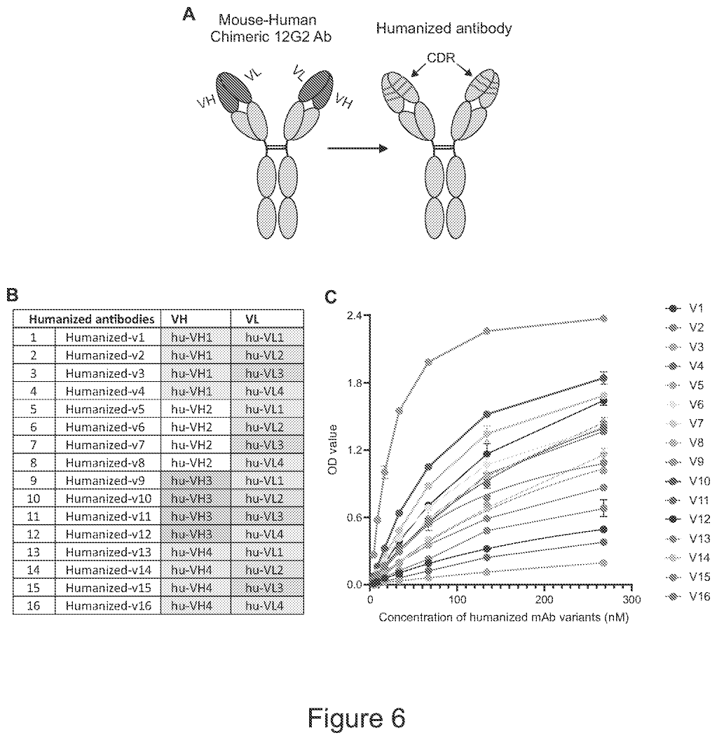

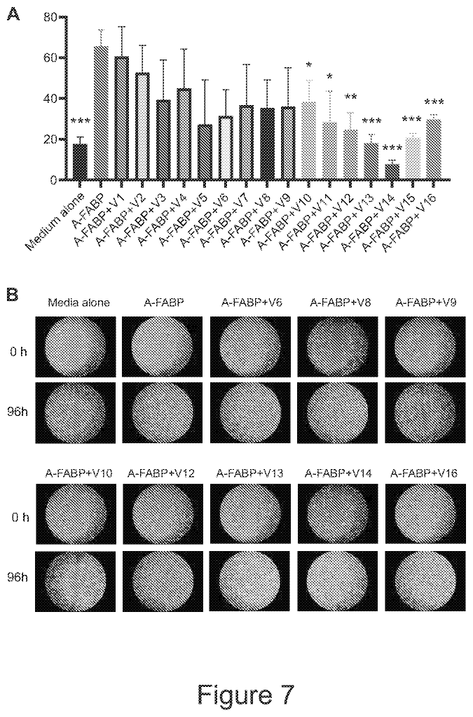

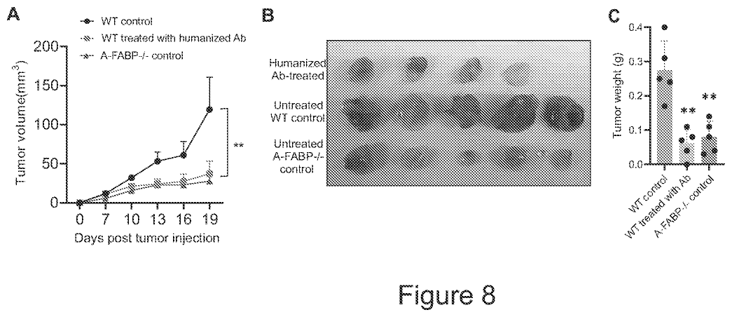

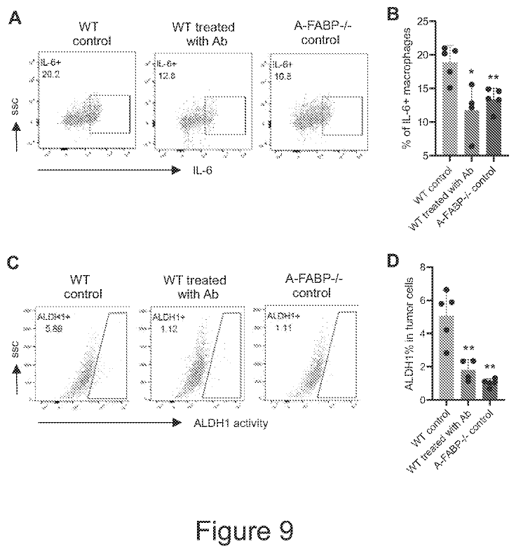

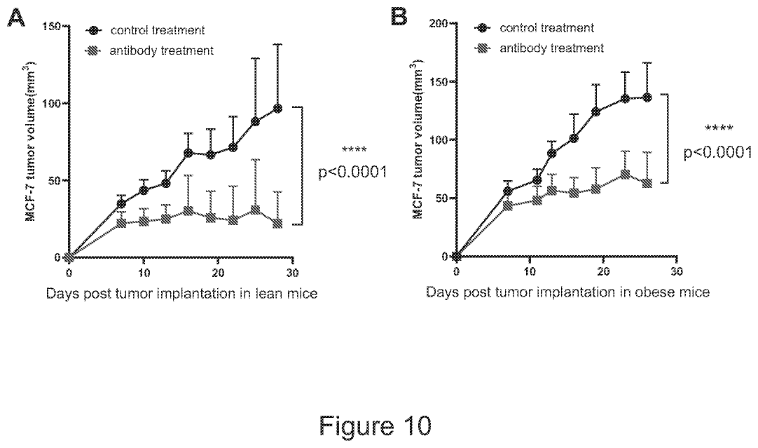

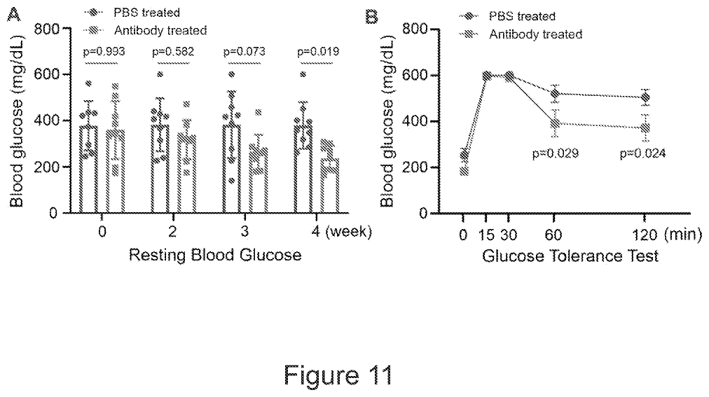

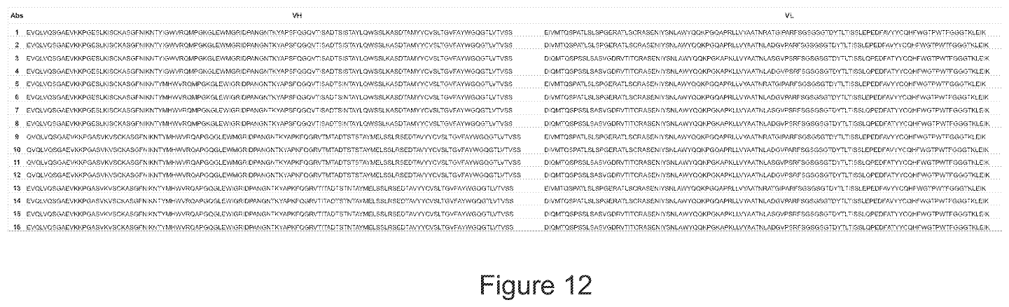

High expression of A-FABP was significantly associated with low survival of BC patients (n=596) (FIG. 1A), suggesting a pro-tumor function of A-FABP. As A-FABP is primarily expressed in adipocytes and macrophages, A-FABP expression in the stroma of BC tissues was analyzed using GEO dataset GSE9014. A-FABP was found to be significantly upregulated in BC stromal tissues independent of BC subtypes (FIG. 1). To verify the results from online public databases, A-FABP expression was measured in normal and malignant breast tissues (FIG. 1C). Compared to normal breast tissues, significantly more A-FABP positive cells were accumulated in the stroma of BC tissues regardless of ER status (FIG. 1D). These data suggest that high expression of A-FABP in the tumor stroma is associated with BC risk.

If A-FABP upregulation was associated with BC risk, A-FABP deficiency might inhibit BC development. To this end, A-FABP deficient (A-FABP^{-/-}) mouse models were used to assess the impact of A-FABP deficiency on mammary tumor growth and metastasis. E0771 tumor cells derived from a C57BL/6 mouse mammary adenocarcinoma were orthotopically injected into the mammary fat pads of A-FABP^{-/-} mice and their WT littermates (C57BL/6 background). E0771 tumors grew much slower in A-FABP^{-/-} mice than in WT mice (FIG. 2A). A-FABP deficiency also significantly reduced E0771 cell-derived lung metastasis (FIG. 2B). To determine whether host expression A-FABP represents a general mechanism promoting other obesity-associated tumor risk, tumor growth and metastasis were evaluated in WT and A-FABP^{-/-} mice using different types of tumor cells, including mammary tumor cells (MMT 060562) and colon cancer cells (MC38). Similar to the E0771 model, both types of tumors exhibited significant decreases in tumor growth and lung metastasis when A-FABP was deficient (FIG. 2C-2F). These data suggest that host-derived A-FABP plays a critical role in promoting obesity-associated tumor risk.

Accumulating evidence suggests that serum levels of A-FABP are markedly increased in obese mice and humans. In our studies, circulating A-FABP levels in a cohort of 84 women without BC were measured. Serum A-FABP levels were positively associated with body mass index (BMI) (FIG. 3A), suggesting an obesity associated increase of circulating A-FABP levels. In another independent cohort of 285 patients with or without BC, A-FABP levels were not only elevated in obese women without BC (FIG. 3B), but more significantly increased in obese women with BC (FIG. 3C). These results suggest that Western diet-induced obesity may contribute to BC risk by increasing soluble levels of A-FABP in the circulation.

Obesity elevates circulating levels of A-FABP, which directly targets BC cells to enhance tumor initiation. Inhibition of A-FABP activity by blocking monoclonal antibodies (mAb) may reduce BC risk. Mice were immunized with human A-FABP (SEQ ID NO:178) and multiple monoclonals were identified that bound to both murine and human A-FABP antigens. Among these selected anti-A-FABP clones, treatment with antibodies from 12G2 clone (SEQ ID Nos. 168 and 169), but not PBS or 6H10 clone, significantly inhibited E0771 mammary tumor growth in vivo (FIG. 4A). Chimeric mAb were prepared with variable (V) regions of the antibody from 12G2 clone and human IgG1 constant (C) regions (kappa). Interestingly, treatment of SCID mice with this chimeric mAb dramatically inhibited human BC MCF-7 tumor growth in vivo (FIG. 4B). Given the success of mAb-based cancer immunotherapy (e.g., anti-PD-1/PD-L1), the data suggest that targeting A-FABP with blocking mAb represents a strategy for obesity-associated cancer treatment.

Besides increasing the risk for cancer, obesity is also known to be associated with other inflammatory diseases, including type 2 diabetes, atherosclerosis, heart disease, etc. To test the efficacy of anti-A-FABP antibodies, diabetic db/db mice were treated with chimeric anti-A-FABP antibodies for two weeks, and serum levels of cytokines were measured. Chimeric 12G2 antibody treatment significantly inhibited proinflammatory cytokine IL-6 production in diabetic mice (FIG. 5), suggesting that blocking A-FABP activity with anti-A-FABP antibody reduces obesity-associated chronic inflammation in vivo. Using the ob/ob diabetic mouse model, it was demonstrated the ob/ob mice treated with humanized anti-A-FABP antibody significantly reduced rest levels of glucose in the circulation (FIG. 11A). Moreover, after exogenous glucose administration, antibody-treated ob/ob mice exhibited significantly improved glucose tolerance when compared to PBS-treated mice (FIG. 11B). These data clearly indicate that humanized anti-A-FABP mAb effectively improved glucose metabolism.

To summarize, data indicate that treatment with anti-A-FABP antibodies represent a strategy for obesity-associated diseases, including different types of cancer and diabetes.

### Example 2

Previous studies have demonstrated that chimeric anti-A-FABP antibody 12G2 inhibited human MCF-7 tumor growth in a xenograft model (FIG. 4). Moreover, treatment with chimeric 12G2 antibody significantly inhibited obesity associated pro-inflammatory cytokine IL-6 production (FIG. 5). These data suggest that this unique 12G2 clone represents a very promising therapeutic antibody. The complementarity determining regions (CDR) of the 12G2 antibody were identified. Through the molecular modeling and in silico humanization processes, 4 humanized VH and VL sequences (SEQ ID Nos. 170-177) have been produced by grafting select CDR amino acids from 12G2 parental sequences onto the closest matching human acceptor germline sequences, which gives a possible 16 humanized antibodies (FIG. 6B). With this panel of purified variant antibodies, their antigen binding affinity is measured by ELISA (FIG. 6C). Variant antibodies with high affinity to A-FABP are selected for in vitro tests, which include MCF-7 cell colony formation assay (FIG. 7A) and MCF-7 wound-healing migration assay (FIG. 7B). Variants with consistent activities in vitro are selected for antibody treatment in vivo. As shown in FIG. 8, treatment of E077 tumor-bearing mice with a selected humanized anti-A-FABP antibody significantly inhibits tumor growth as compared to PBS-treated control mice. Tumor size and weight in humanized antibody treated group were similar as these in A-FABP^{-/-} mice (FIG. 8B, 8C), suggesting that the antibody totally blocks A-FABP activity in vivo. Further in vivo functional assays demonstrated that anti-A-FABP antibody inhibits tumor growth at least via two mechanisms: 1) inhibition of IL-6 production from tumor infiltrated macrophages (FIG. 9A, 9B); 2) reducing tumor stemness by inhibition of tumor cell ALDH1 activity (FIG. 9C, 9D). Moreover, the therapeutic efficacy of the humanized anti-A-FABP antibody was shown using the MCF-7 xenograft mouse model in both lean SCID mice and obese SCID mice (FIG. 10). Collectively, these data clearly implicate that humanized anti-A-FABP antibodies represent novel strategies for clinical treatment of BC and other obesity-associated diseases.

### Example 3

### Exemplary Antibody Sequences (Exemplary CDRs are Underlined in Antibodies 1-16) Parental 12G2 Antibody

VH:
(SEQ ID NO: 168)
EVQLQQSVAELVRPGASVKLSCTASGFNIKNTYMHWVKQRPEQGLEWIGR
IDPANGNTKYAPKFQGKATITADTSSNTAYLQLSSLTSEDTAIYYCVSLT
GVFAYWGQGTLVTVSA.
VL:
(SEQ ID NO: 169)
DIQMTQSPASLSVSVGETVTITCRASENIYSNLAWYQQKQGKSPQLLVYA
ATNLADGVPSRFSGSGSGTQYSLKINSLQSEDFGSYYCQHFWGTPWTFGG
GTKLEIK

### Human Framework

VH1:
(SEQ ID NO: 170)
EVQLVQSGAEVKKPGASVKVSCKASGFNIKNTYMHWVRQAPGQGLEWIGR
IDPANGNTKYAPKFQGRVTITADTSTNTAYMELSSLRSEDTAVYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VH2:
(SEQ ID NO: 171)
QVQLVQSGAEVKKPGASVKVSCKASGFNIKNTYMHWVRQAPGQGLEWMGR
IDPANGNTKYAPKFQGRVTMTADTSTSTAYMELSSLRSEDTAVYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VH3:
(SEQ ID NO: 172)
EVQLVQSGAEVKKPGESLKISCKASGFNIKNTYMHWVRQMPGKGLEWIGR
IDPANGNTKYAPSFQGQVTISADTSINTAYLQWSSLKASDTAMYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VH4:
(SEQ ID NO: 173)
EVQLVQSGAEVKKPGESLKISCKASGFNIKNTYIGWVRQMPGKGLEWMGR
IDPANGNTKYAPSFQGQVTISADTSISTAYLQWSSLKASDTAMYYCVSLT
GVFAYWGQGTLVTVSS
VL1:
(SEQ ID NO: 174)
DIQMTQSPSSLSASVGDRVTITCRASENIYSNLAWYQQKPGKAPKLLVYA
ATNLADGVPSRFSGSGSGTDYTLTISSLQPEDFATYYCQHFWGTPWTFGG
GTKLEIK
or a polypeptide with at least 80%, 85%, 90%, 92%,
94%, 95%, 96%, 97%, 98%, or 99% amino acid
sequence identity thereto.
VL2:
(SEQ ID NO: 175)
DIQMTQSPSSLSASVGDRVTITCRASENIYSNLAWYQQKPGKAPKLLVYA
ATNLASGVPSRFSGSGSGTDYTLTISSLQPEDFATYYCQHFWGTPWTFGG
GTKLEIK
or a polypeptide with at least 80%, 85%, 90%, 92%,
94%, 95%, 96%, 97%, 98%, or 99% amino acid
sequence identity thereto.
VL3:
(SEQ ID NO: 176)
DIVMTQSPATLSLSPGERATLSCRASENIYSNLAWYQQKPGQAPRLLVYA
ATNLADGVPARFSGSGSGTDYTLTISSLEPEDFAVYYCQHFWGTPWTFGG
GTKLEIK
or a polypeptide with at least 80%, 85%, 90%, 92%,
94%, 95%, 96%, 97%, 98%, or 99% amino acid
sequence identity thereto.
VL4:
(SEQ ID NO: 177)
EIVMTQSPATLSLSPGERATLSCRASENIYSNLAWYQQKPGQAPRLLVYA
ATNRATGIPARFSGSGSGTDYTLTISSLEPEDFAVYYCQHFWGTPWTFGG
GTKLEIK
or a polypeptide with at least 80%, 85%, 90%, 92%,
94%, 95%, 96%, 97%, 98%, or 99% amino acid
sequence identity thereto.

### Antibody 1

VH:
(SEQ ID NO: 7)
EVQLVQSGAEVKKPGESLKISCKASGFNIKNTYIGWVRQMPGKGLEWMGR
IDPANGNTKYAPSFQGQVTISADTSISTAYLQWSSLKASDTAMYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VL:
(SEQ ID NO: 8)
EIVMTQSPATLSLSPGERATLSCRASENIYSNLAWYQQKPGQAPRLLVYA
ATNRATGIPARFSGSGSGTDYTLTISSLEPEDFAVYYCQHFWGTPWTFGG
GTKLEIK
or a polypeptide with at least 80%, 85%, 90%, 92%,
94%, 95%, 96%, 97%, 98%, or 99% amino acid
sequence identity thereto.

### Antibody 2

VH:
(SEQ ID NO: 9)
EVQLVQSGAEVKKPGESLKISCKASGFNIKNTYIGWVRQMPGKGLEWMGR
IDPANGNTKYAPSFQGQVTISADTSISTAYLQWSSLKASDTAMYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VL:
(SEQ ID NO: 10)
DIVMTQSPATLSLSPGERATLSCRASENIYSNLAWYQQKPGQAPRLLVYA
ATNLADGVPARFSGSGSGTDYTLTISSLEPEDFAVYYCQHFWGTPWTFGG
GTKLEIK
or a polypeptide with at least 80%, 85%, 90%, 92%,
94%, 95%, 96%, 97%, 98%, or 99% amino acid
sequence identity thereto.

### Antibody 3

VH:
(SEQ ID NO: 11)
EVQLVQSGAEVKKPGESLKISCKASGFNIKNTYIGWVRQMPGKGLEWMGR
IDPANGNTKYAPSFQGQVTISADTSISTAYLQWSSLKASDTAMYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VL:
(SEQ ID NO: 12)
DIQMTQSPSSLSASVGDRVTITCRASENIYSNLAWYQQKPGKAPKLLVYA
ATNLASGVPSRFSGSGSGTDYTLTISSLQPEDFATYYCQHFWGTPWTFGG
GTKLEIK
or a polypeptide with at least 80%, 85%, 90%, 92%,
94%, 95%, 96%, 97%, 98%, or 99% amino acid
sequence identity thereto.

### Antibody 4

VH:
(SEQ ID NO: 13)
EVQLVQSGAEVKKPGESLKISCKASGFNIKNTYIGWVRQMPGKGLEWMGR
IDPANGNTKYAPSFQGQVTISADTSISTAYLQWSSLKASDTAMYYCVSLT
GVFAYWGQGTLVTVSS
or a polypeptide with at least 80%, 85%, 90%, 92%,
94%, 95%, 96%, 97%, 98%, or 99% amino acid
sequence identity thereto.
VL
(SEQ ID NO: 14)
DIQMTQSPSSLSASVGDRVTITCRASENIYSNLAWYQQKPGKAPKLLVYA
ATNLADGVPSRFSGSGSGTDYTLTISSLQPEDFATYYCQHFWGTPWTFGG
GTKLEIK
or a polypeptide with at least 80%, 85%, 90%, 92%,
94%, 95%, 96%, 97%, 98%, or 99% amino acid
sequence identity thereto.

### Antibody 5

VH:
(SEQ ID NO: 15)
EVQLVQSGAEVKKPGESLKISCKASGFNIKNTYMHWVRQMPGKGLEWIGRIDPANG
NTKYAPSFQGQVTISADTSINTAYLQWSSLKASDTAMYYCVSLTGVFAYWGQGTLV
TVSS
or
a polypeptide with at least 80%, 85%, 90%, 92%, 94%, 95%,
96%, 97%, 98%, or 99% amino acid sequence identity thereto.
VL:
(SEQ ID NO: 16)
EIVMTQSPATLSLSPGERATLSCRASENIYSNLAWYQQKPGQAPRLLVYAATNRATG
IPARFSGSGSGTDYTLTISSLEPEDFAVYYCQHFWGTPWTFGGGTKLEIK
or
a polypeptide with at least 80%, 85%, 90%, 92%, 94%, 95%, 96%,
97%, 98%, or 99% amino acid sequence identity thereto.

### Antibody 6

VH:
(SEQ ID NO: 17)
EVQLVQSGAEVKKPGESLKISCKASGFNIKNTYMHWVRQMPGKGLEWIGRIDPANG
NTKYAPSFQGQVTISADTSINTAYLQWSSLKASDTAMYYCVSLTGVFAYWGQGTLV
TVSS
or
a polypeptide with at least 80%, 85%, 90%, 92%, 94%, 95%,
96%, 97%, 98%, or 99% amino acid sequence identity thereto.
VL:
(SEQ ID NO: 18)
DIVMTQSPATLSLSPGERATLSCRASENIYSNLAWYQQKPGQAPRLLVYAATNLADG
VPARFSGSGSGTDYTLTISSLEPEDFAVYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 7

VH:
(SEQ ID NO: 19)
EVQLVQSGAEVKKPGESLKISCKASGFNIKNTYMHWVRQMPGKGLEWIGRIDPANG
NTKYAPSFQGQVTISADTSINTAYLQWSSLKASDTAMYYCVSLTGVFAYWGQGTLV
TVSS
or
a polypeptide with at least 80%, 85%, 90%, 92%, 94%, 95%,
96%, 97%, 98%, or 99% amino acid sequence identity thereto.
VL:
(SEQ ID NO: 20)
DIQMTQSPSSLSASVGDRVTITCRASENIYSNLAWYQQKPGKAPKLLVYAATNLASG
VPSRFSGSGSGTDYTLTISSLQPEDFATYYCQHFWGTPWTFGGGTKLEIK
or
a polypeptide with at least 80%, 85%, 90%, 92%, 94%, 95%, 96%, 97%, 98%,
or 99% amino acid sequence identity thereto.

### Antibody 8

VH:
(SEQ ID NO: 21)
EVQLVQSGAEVKKPGESLKISCKASGFNIKNTYMHWVRQMPGKGLEWIGRIDPANG
NTKYAPSFQGQVTISADTSINTAYLQWSSLKASDTAMYYCVSLTGVFAYWGQGTLV
TVSS
or
a polypeptide with at least 80%, 85%, 90%, 92%, 94%, 95%,
96%, 97%, 98%, or 99% amino acid sequence identity thereto.
VL:
(SEQ ID NO: 22)
DIQMTQSPSSLSASVGDRVTITCRASENIYSNLAWYQQKPGKAPKLLVYAATNLADG
VPSRFSGSGSGTDYTLTISSLQPEDFATYYCQHFWGTPWTFGGGTKLEIK
or
a polypeptide with at least 80%, 85%, 90%, 92%, 94%, 95%, 96%, 97%,
98%, or 9% amino acid sequence identity thereto.

### Antibody 9

VH:
(SEQ ID NO: 23)
QVQLVQSGAEVKKPGASVKVSCKASGFNIKNTYMHWVRQAPGQGLEWMGRIDPA
NGNTKYAPKFQGRVTMTADTSTSTAYMELSSLRSEDTAVYYCVSLTGVFAYWGQG
TLVTVSS
or
a polypeptide with at least 80%, 85%, 90%, 92%, 94%, 95%,
96%, 97%, 98%, or 99% amino acid sequence identity thereto.
VL:
(SEQ ID NO: 24)
EIVMTQSPATLSLSPGERATLSCRASENIYSNLAWYQQKPGQAPRLLVYAATNRATG
IPARFSGSGSGTDYTLTISSLEPEDFAVYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 10

VH:
(SEQ ID NO: 25)
VQLVQSGAEVKKPGASVKVSCKASGFNIKNTYMHWVRQAPGQGLEWMGRIDPAN
GNTKYAPKFQGRVTMTADTSTSTAYMELSSLRSEDTAVYYCVSLTGVFAYWGQGT
LVTVSS
or
a polypeptide with at least 80%, 85%, 90%, 92%, 94%, 95%,
96%, 97%, 98%, or 99% amino acid sequence identity thereto.
VL:
(SEQ ID NO: 26)
DIVMTQSPATLSLSPGERATLSCRASENIYSNLAWYQQKPGQAPRLLVYAATNLADG
VPARFSGSGSGTDYTLTISSLEPEDFAVYYCQHFWGTPWTFGGGTKLEIK
or
a polypeptide with at least 80%, 85%, 90%, 92%, 94%, 95%, 96%, 97%,
98%, or 99% amino acid sequence identity thereto.

Antibody 11

VH:
(SEQ ID NO: 27)
QVQLVQSGAEVKKPGASVKVSCKASGFNIKNTYMHWVRQAPGQGLEWMGRIDPA
NGNTKYAPKFQGRVTMTADTSTSTAYMELSSLRSEDTAVYYCVSLTGVFAYWGQG
TLVTVSS
or
a polypeptide with at least 80%, 85%, 90%, 92%, 94%, 95%,
96%, 97%, 98%, or 99% amino acid sequence identity thereto.
VL:
(SEQ ID NO: 28)
DIQMTQSPSSLSASVGDRVTITCRASENIYSNLAWYQQKPGKAPKLLVYAATNLASG
VPSRFSGSGSGTDYTLTISSLQPEDFATYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 12

VH:
(SEQ ID NO: 29)
QVQLVQSGAEVKKPGASVKVSCKASGFNIKNTYMHWVRQAPGQGLEWMGRIDPA
NGNTKYAPKFQGRVTMTADTSTSTAYMELSSLRSEDTAVYYCVSLTGVFAYWGQG
TLVTVSS
or
a polypeptide with at least 80%, 85%, 90%, 92%, 94%, 95%,
96%, 97%, 98%, or 99% amino acid sequence identity thereto.
VL:
(SEQ ID NO: 30)
DIQMTQSPSSLSASVGDRVTITCRASENIYSNLAWYQQKPGKAPKLLVYAATNLADG
VPSRFSGSGSGTDYTLTISSLQPEDFATYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 13

VH:
(SEQ ID NO: 31)
EVQLVQSGAEVKKPGASVKVSCKASGFNIKNTYMHWVRQAPGQGLEWIGRIDPAN
GNTKYAPKFQGRVTITADTSTNTAYMELSSLRSEDTAVYYCVSLTGVFAYWGQGTL
VTVSS
or
a polypeptide with at least 80%, 85%, 90%, 92%, 94%, 95%,
96%, 97%, 98%, or 99% amino acid sequence identity thereto.
VL:
(SEQ ID NO: 32)
EIVMTQSPATLSLSPGERATLSCRASENIYSNLAWYQQKPGQAPRLLVYAATNRATG
IPARFSGSGSGTDYTLTISSLEPEDFAVYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 14

VH:
(SEQ ID NO: 33)
EVQLVQSGAEVKKPGASVKVSCKASGFNIKNTYMHWVRQAPGQGLEWIGRIDPAN
GNTKYAPKFQGRVTITADTSTNTAYMELSSLRSEDTAVYYCVSLTGVFAYWGQGTL
VTVSS
or
a polypeptide with at least 80%, 85%, 90%, 92%, 94%, 95%,
96%, 97%, 98%, or 99% amino acid sequence identity thereto.
VL:
(SEQ ID NO: 34)
DIVMTQSPATLSLSPGERATLSCRASENIYSNLAWYQQKPGQAPRLLVYAATNLADG
VPARFSGSGSGTDYTLTISSLEPEDFAVYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 15

VH:
(SEQ ID NO: 35)
EVQLVQSGAEVKKPGASVKVSCKASGFNIKNTYMHWVRQAPGQGLEWIGRIDPAN
GNTKYAPKFQGRVTITADTSTNTAYMELSSLRSEDTAVYYCVSLTGVFAYWGQGTL
VTVSS
or
a polypeptide with at least 80%, 85%, 90%, 92%, 94%, 95%,
96%, 97%, 98%, or 99% amino acid sequence identity thereto.
VL:
(SEQ ID NO: 36)
DIQMTQSPSSLSASVGDRVTITCRASENIYSNLAWYQQKPGKAPKLLVYAATNLASG
VPSRFSGSGSGTDYTLTISSLQPEDFATYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Antibody 16

VH:
(SEQ ID NO: 37)
EVQLVQSGAEVKKPGASVKVSCKASGFNIKNTYMHWVRQAPGQGLEWIGRIDPAN
GNTKYAPKFQGRVTITADTSTNTAYMELSSLRSEDTAVYYCVSLTGVFAYWGQGTL
VTVSS
or
a polypeptide with at least 80%, 85%, 90%, 92%, 94%, 95%,
96%, 97%, 98%, or 99% amino acid sequence identity thereto.
VL:
(SEQ ID NO: 38)
DIQMTQSPSSLSASVGDRVTITCRASENIYSNLAWYQQKPGKAPKLLVYAATNLADG
VPSRFSGSGSGTDYTLTISSLQPEDFATYYCQHFWGTPWTFGGGTKLEIK
or
a polypeptide with at least 80%, 85%, 90%, 92%, 94%, 95%, 96%, 97%,
98%, or 99% amino acid sequence identity thereto.

### Exemplary CDRs for a Heavy Chain Variable Region Comprise:

(SEQ ID NO: 1)
GFNIKNTY
or
a sequence with 1, 2, 3, 4, or 5 substitutions
(SEQ ID NO: 2)
IDPANGNT
or
a sequence with 1, 2, 3, 4, or 5 substitutions,
and/or
(SEQ ID NO: 3)
VSLTGVFAY
or
a sequence with 1, 2, 3, 4, or 5 substitutions.

### Exemplary CDRs for a Light Chain Variable Region Comprise:

(SEQ ID NO: 4)
ENIYSN
or
a sequence with 1, 2, or 3 substitutions
AAT
or
a sequence with 1 or 2 substitutions,
and/or
(SEQ ID NO: 6)
QHFWGTPWT
or
a sequence with 1, 2, 3, 4, or 5 substitutions.

### Exemplary A-FABP (FABP4) Sequence:

(SEQ ID NO: 178)
MCDAFVGTWKLVSSENFDDYMKEVGVGFATRKVAGMAKPNMIISVNGDV
ITIKSESTFKNTEISFILGQEFDEVTADDRKVKSTITLDGGVLVHVQKW
DGKSTTIKRKREDDKLVVECVMKGVTSTRVYERA

Exemplary Framework Sequences that Flank One or More Exemplary CDRs:

EVQLVQSGAEVKKPGESLKISCKAS (SEQ ID NO:40) or a polypeptide with at least 80%, 85%, 90%, 92%, 94%, 95%, 96%, 97%, 98%, or 99% amino acid sequence identity thereto. IGWVRQMPGKGLEWMGR (SEQ ID NO:41) or a polypeptide with at least 80%, 85%, 90%, 92%, 94%, 95%, 96%, 97%, 98%, or 99% amino acid sequence identity thereto.

KYAPSFQGQVTISADTSISTAYLQWSSLKASDTAMYYC (SEQ ID NO:42) or a polypeptide with at least 80%, 85%, 90%, 92%, 94%, 95%, 96%, 97%, 98%, or 99% amino acid sequence identity thereto.

YWGQGTLVTVSS (SEQ ID NO:43) or a polypeptide with at least 80%, 85%, 90%, 92%, 94%, 95%, 96%, 97%, 98%, or 99% amino acid sequence identity thereto.

EIVMTQSPATLSLSPGERATLSCRAS (SEQ ID NO:44) or a polypeptide with at least 80%, 85%, 90%, 92%, 94%, 95%, 96%, 97%, 98%, or 99% amino acid sequence identity thereto.

LAWYQQKPGQAPRLLVY(SEQ ID NO:45) or a polypeptide with at least 80%, 85%, 90%, 92%, 94%, 95%, 96%, 97%, 98%, or 99% amino acid sequence identity thereto.

NRATGIPARFSGSGSGTDYTLTISSLEPEDFAVYYC (SEQ ID NO:46) or a polypeptide with at least 80%, 85%, 90%, 92%, 94%, 95%, 96%, 97%, 98%, or 99% amino acid sequence identity thereto.

FGGGTKLEIK (SEQ ID NO:47) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:48) or a polypeptide with at least 80%, 85%, 90%, 92%, 94%, 95%, 96%, 97%, 98%, or 99% amino acid sequence identity thereto.

IGWVRQMPGKGLEWMGR (SEQ ID NO:49) or a polypeptide with at least 80%, 85%, 90%, 92%, 94%, 95%, 96%, 97%, 98%, or 99% amino acid sequence identity thereto.

KYAPSFQGQVTISADTSISTAYLQWSSLKASDTAMYYC (SEQ ID NO:50) or a polypeptide with at least 80%, 85%, 90%, 92%, 94%, 95%, 96%, 97%, 98%, or 99% amino acid sequence identity thereto.

WGQGTLVTVSS (SEQ ID NO:51) or a polypeptide with at least 80%, 85%, 90%, 92%, 94%, 95%, 96%, 97%, 98%, or 99% amino acid sequence identity thereto.

DIVMTQSPATLSLSPGERATLSCRAS (SEQ ID NO:52) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:53) or a polypeptide with at least 80%, 85%, 90%, 92%, 94%, 95%, 96%, 97%, 98%, or 99% amino acid sequence identity thereto.

NLADGVPARFSGSGSGTDYTLTISSLEPEDFAVYYC (SEQ ID NO:54) FGGGTKLEIK (SEQ ID NO:55) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:56) or a polypeptide with at least 80%, 85%, 90%, 92%, 94%, 95%, 96%, 97%, 98%, or 99% amino acid sequence identity thereto.

IGWVRQMPGKGLEWMGR (SEQ ID NO:57) or a polypeptide with at least 80%, 85%, 90%, 92%, 94%, 95%, 96%, 97%, 98%, or 99% amino acid sequence identity thereto.

KYAPSFQGQVTISADTSISTAYLQWSSLKASDTAMYYC (SEQ ID NO:58) or a polypeptide with at least 80%, 85%, 90%, 92%, 94%, 95%, 96%, 97%, 98%, or 99% amino acid sequence identity thereto.

WGQGTLVTVSS (SEQ ID NO:59) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:60) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:61) or a polypeptide with at least 80%, 85%, 90%, 92%, 94%, 95%, 96%, 97%, 98%, or 99% amino acid sequence identity thereto.

NLASGVPSRFSGSGSGTDYTLTISSLQPEDFATYYC (SEQ ID NO:62) or a polypeptide with at least 80%, 85%, 90%, 92%, 94%, 95%, 96%, 97%, 98%, or 99% amino acid sequence identity thereto.

FGGGTKLEIK (SEQ ID NO:63) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:64) or a polypeptide with at least 80%, 85%, 90%, 92%, 94%, 95%, 96%, 97%, 98%, or 99% amino acid sequence identity thereto.

IGWVRQMPGKGLEWMGR (SEQ ID NO:65) or a polypeptide with at least 80%, 85%, 90%, 92%, 94%, 95%, 96%, 97%, 98%, or 99% amino acid sequence identity thereto.

KYAPSFQGQVTISADTSISTAYLQWSSLKASDTAMYYC (SEQ ID NO:66) or a polypeptide with at least 80%, 85%, 90%, 92%, 94%, 95%, 96%, 97%, 98%, or 99% amino acid sequence identity thereto.

WGQGTLVTVSS (SEQ ID NO:67) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:68) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:69) or a polypeptide with at least 80%, 85%, 90%, 92%, 94%, 95%, 96%, 97%, 98%, or 99% amino acid sequence identity thereto.

NLADGVPSRFSGSGSGTDYTLTISSLQPEDFATYYC (SEQ ID NO:70) or a polypeptide with at least 80%, 85%, 90%, 92%, 94%, 95%, 96%, 97%, 98%, or 99% amino acid sequence identity thereto.

FGGGTKLEIK (SEQ ID NO:71) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:72) or a polypeptide with at least 80%, 85%, 90%, 92%, 94%, 95%, 96%, 97%, 98%, or 99% amino acid sequence identity thereto.

MHWVRQMPGKGLEWIGR (SEQ ID NO:73) or a polypeptide with at least 80%, 85%, 90%, 92%, 94%, 95%, 96%, 97%, 98%, or 99% amino acid sequence identity thereto.

KYAPSFQGQVTISADTSINTAYLQWSSLKASDTAMYYC (SEQ ID NO:74) or a polypeptide with at least 80%, 85%, 90%, 92%, 94%, 95%, 96%, 97%, 98%, or 99% amino acid sequence identity thereto.

WGQGTLVTVSS (SEQ ID NO:75) or a polypeptide with at least 80%, 85%, 90%, 92%, 94%, 95%, 96%, 97%, 98%, or 99% amino acid sequence identity thereto.

EIVMTQSPATLSLSPGERATLSCRAS (SEQ ID NO:76) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:77) or a polypeptide with at least 80%, 85%, 90%, 92%, 94%, 95%, 96%, 97%, 98%, or 99% amino acid sequence identity thereto.

NRATGIPARFSGSGSGTDYTLTISSLEPEDFAVYYC (SEQ ID NO:78) or a polypeptide with at least 80%, 85%, 90%, 92%, 94%, 95%, 96%, 97%, 98%, or 99% amino acid sequence identity thereto.

FGGGTKLEIK (SEQ ID NO:79) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:80) or a polypeptide with at least 80%, 85%, 90%, 92%, 94%, 95%, 96%, 97%, 98%, or 99% amino acid sequence identity thereto.

MHWVRQMPGKGLEWIGR (SEQ ID NO:81) or a polypeptide with at least 80%, 85%, 90%, 92%, 94%, 95%, 96%, 97%, 98%, or 99% amino acid sequence identity thereto. KYAPSFQGQVTISADTSINTAYLQWSSLKASDTAMYYC (SEQ ID NO:82) or a polypeptide with at least 80%, 85%, 90%, 92%, 94%, 95%, 96%, 97%, 98%, or 99% amino acid sequence identity thereto.

WGQGTLVTVSS (SEQ ID NO:83) or a polypeptide with at least 80%, 85%, 90%, 92%, 94%, 95%, 96%, 97%, 98%, or 99% amino acid sequence identity thereto.

DIVMTQSPATLSLSPGERATLSCRAS (SEQ ID NO:84) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:85) or a polypeptide with at least 80%, 85%, 90%, 92%, 94%, 95%, 96%, 97%, 98%, or 99% amino acid sequence identity thereto.

NLADGVPARFSGSGSGTDYTLTISSLEPEDFAVYYC (SEQ ID NO:86) or a polypeptide with at least 80%, 85%, 90%, 92%, 94%, 95%, 96%, 97%, 98%, or 99% amino acid sequence identity thereto.

FGGGTKLEIK (SEQ ID NO:87) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:88) or a polypeptide with at least 80%, 85%, 90%, 92%, 94%, 95%, 96%, 97%, 98%, or 99% amino acid sequence identity thereto.

MHWVRQMPGKGLEWIGR (SEQ ID NO:89) or a polypeptide with at least 80%, 85%, 90%, 92%, 94%, 95%, 96%, 97%, 98%, or 99% amino acid sequence identity thereto.

KYAPSFQGQVTISADTSINTAYLQWSSLKASDTAMYYC (SEQ ID NO:90) WGQGTLVTVSS (SEQ ID NO:91) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:92) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:93) or a polypeptide with at least 80%, 85%, 90%, 92%, 94%, 95%, 96%, 97%, 98%, or 99% amino acid sequence identity thereto.

NLASGVPSRFSGSGSGTDYTLTISSLQPEDFATYYC (SEQ ID NO:94) or a polypeptide with at least 80%, 85%, 90%, 92%, 94%, 95%, 96%, 97%, 98%, or 99% amino acid sequence identity thereto.

FGGGTKLEIK (SEQ ID NO:95) or a polypeptide with at least 80%, 85%, 90%, 92%, 94%, 95%, 96%, 97%, 98%, or 99% amino acid sequence identity thereto.

EVQLVQSGAEVKKPGESLKISCKAS (SEQ ID NO:97) or a polypeptide with at least 80%, 85%, 90%, 92%, 94%, 95%, 96%, 97%, 98%, or 99% amino acid sequence identity thereto.

MHWVRQMPGKGLEWIGR (SEQ ID NO:98) or a polypeptide with at least 80%, 85%, 90%, 92%, 94%, 95%, 96%, 97%, 98%, or 99% amino acid sequence identity thereto.

KYAPSFQGQVTISADTSINTAYLQWSSLKASDTAMYYC (SEQ ID NO:99) or a polypeptide with at least 80%, 85%, 90%, 92%, 94%, 95%, 96%, 97%, 98%, or 99% amino acid sequence identity thereto.

WGQGTLVTVSS (SEQ ID NO:100) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:101) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:102) or a polypeptide with at least 80%, 85%, 90%, 92%, 94%, 95%, 96%, 97%, 98%, or 99% amino acid sequence identity thereto.

NLADGVPSRFSGSGSGTDYTLTISSLQPEDFATYYC (SEQ ID NO:103) or a polypeptide with at least 80%, 85%, 90%, 92%, 94%, 95%, 96%, 97%, 98%, or 99% amino acid sequence identity thereto.

FGGGTKLEIK (SEQ ID NO:104) or a polypeptide with at least 80%, 85%, 90%, 92%, 94%, 95%, 96%, 97%, 98%, or 99% amino acid sequence identity thereto.

QVQLVQSGAEVKKPGASVKVSCKAS (SEQ ID NO:105) or a polypeptide with at least 80%, 85%, 90%, 92%, 94%, 95%, 96%, 97%, 98%, or 99% amino acid sequence identity thereto.

MHWVRQAPGQGLEWMGR (SEQ ID NO:106) or a polypeptide with at least 80%, 85%, 90%, 92%, 94%, 95%, 96%, 97%, 98%, or 99% amino acid sequence identity thereto.

KYAPKFQGRVTMTADTSTSTAYMELSSLRSEDTAVYYC (SEQ ID NO:107) or a polypeptide with at least 80%, 85%, 90%, 92%, 94%, 95%, 96%, 97%, 98%, or 99% amino acid sequence identity thereto.

WGQGTLVTVSS (SEQ ID NO:108) or a polypeptide with at least 80%, 85%, 90%, 92%, 94%, 95%, 96%, 97%, 98%, or 99% amino acid sequence identity thereto.

EIVMTQSPATLSLSPGERATLSCRAS (SEQ ID NO:109) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:110) or a polypeptide with at least 80%, 85%, 90%, 92%, 94%, 95%, 96%, 97%, 98%, or 99% amino acid sequence identity thereto.

NRATGIPARFSGSGSGTDYTLTISSLEPEDFAVYYC (SEQ ID NO:111) or a polypeptide with at least 80%, 85%, 90%, 92%, 94%, 95%, 96%, 97%, 98%, or 99% amino acid sequence identity thereto.

FGGGTKLEIK (SEQ ID NO:112) or a polypeptide with at least 80%, 85%, 90%, 92%, 94%, 95%, 96%, 97%, 98%, or 99% amino acid sequence identity thereto.

VQLVQSGAEVKKPGASVKVSCKAS (SEQ ID NO:113) or a polypeptide with at least 80%, 85%, 90%, 92%, 94%, 95%, 96%, 97%, 98%, or 99% amino acid sequence identity thereto.

MHWVRQAPGQGLEWMGR (SEQ ID NO:114) or a polypeptide with at least 80%, 85%, 90%, 92%, 94%, 95%, 96%, 97%, 98%, or 99% amino acid sequence identity thereto.

KYAPKFQGRVTMTADTSTSTAYMELSSLRSEDTAVYYC (SEQ ID NO:115) or a polypeptide with at least 80%, 85%, 90%, 92%, 94%, 95%, 96%, 97%, 98%, or 99% amino acid sequence identity thereto.

WGQGTLVTVSS (SEQ ID NO:116) or a polypeptide with at least 80%, 85%, 90%, 92%, 94%, 95%, 96%, 97%, 98%, or 99% amino acid sequence identity thereto.

DIVMTQSPATLSLSPGERATLSCRAS (SEQ ID NO:117) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:118) or a polypeptide with at least 80%, 85%, 90%, 92%, 94%, 95%, 96%, 97%, 98%, or 99% amino acid sequence identity thereto.

NLADGVPARFSGSGSGTDYTLTISSLEPEDFAVYYC (SEQ ID NO:119) or a polypeptide with at least 80%, 85%, 90%, 92%, 94%, 95%, 96%, 97%, 98%, or 99% amino acid sequence identity thereto.

FGGGTKLEIK (SEQ ID NO:120) or a polypeptide with at least 80%, 85%, 90%, 92%, 94%, 95%, 96%, 97%, 98%, or 99% amino acid sequence identity thereto.

QVQLVQSGAEVKKPGASVKVSCKAS (SEQ ID NO:121) or a polypeptide with at least 80%, 85%, 90%, 92%, 94%, 95%, 96%, 97%, 98%, or 99% amino acid sequence identity thereto.

MHWVRQAPGQGLEWMGR (SEQ ID NO:122) or a polypeptide with at least 80%, 85%, 90%, 92%, 94%, 95%, 96%, 97%, 98%, or 99% amino acid sequence identity thereto.

KYAPKFQGRVTMTADTSTSTAYMELSSLRSEDTAVYYC (SEQ ID NO:123) WGQGTLVTVSS (SEQ ID NO:124) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:125) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:126) or a polypeptide with at least 80%, 85%, 90%, 92%, 94%, 95%, 96%, 97%, 98%, or 99% amino acid sequence identity thereto.

NLASGVPSRFSGSGSGTDYTLTISSLQPEDFATYYC_(SEQ ID NO: 127) or a polypeptide with at least 80%, 85%, 90%, 92%, 94%, 95%, 96%, 97%, 98%, or 99% amino acid sequence identity thereto.

GGGTKLEIK (SEQ ID NO:128) or a polypeptide with at least 80%, 85%, 90%, 92%, 94%, 95%, 96%, 97%, 98%, or 99% amino acid sequence identity thereto.

QVQLVQSGAEVKKPGASVKVSCKAS (SEQ ID NO:129) or a polypeptide with at least 80%, 85%, 90%, 92%, 94%, 95%, 96%, 97%, 98%, or 99% amino acid sequence identity thereto.

MHWVRQAPGQGLEWMGR (SEQ ID NO:130) or a polypeptide with at least 80%, 85%, 90%, 92%, 94%, 95%, 96%, 97%, 98%, or 99% amino acid sequence identity thereto.

KYAPKFQGRVTMTADTSTSTAYMELSSLRSEDTAVYYC (SEQ ID NO:131) or a polypeptide with at least 80%, 85%, 90%, 92%, 94%, 95%, 96%, 97%, 98%, or 99% amino acid sequence identity thereto.

WGQGTLVTVSS (SEQ ID NO:132) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:133) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:134) or a polypeptide with at least 80%, 85%, 90%, 92%, 94%, 95%, 96%, 97%, 98%, or 99% amino acid sequence identity thereto.

NLADGVPSRFSGSGSGTDYTLTISSLQPEDFATYYC (SEQ ID NO:135) or a polypeptide with at least 80%, 85%, 90%, 92%, 94%, 95%, 96%, 97%, 98%, or 99% amino acid sequence identity thereto.

FGGGTKLEIK (SEQ ID NO:136) or a polypeptide with at least 80%, 85%, 90%, 92%, 94%, 95%, 96%, 97%, 98%, or 99% amino acid sequence identity thereto.

EVQLVQSGAEVKKPGASVKVSCKAS (SEQ ID NO:137) or a polypeptide with at least 80%, 85%, 90%, 92%, 94%, 95%, 96%, 97%, 98%, or 99% amino acid sequence identity thereto.

MHWVRQAPGQGLEWIGR (SEQ ID NO:138) or a polypeptide with at least 80%, 85%, 90%, 92%, 94%, 95%, 96%, 97%, 98%, or 99% amino acid sequence identity thereto.

KYAPKFQGRVTITADTSTNTAYMELSSLRSEDTAVYYC (SEQ ID NO:139) or a polypeptide with at least 80%, 85%, 90%, 92%, 94%, 95%, 96%, 97%, 98%, or 99% amino acid sequence identity thereto.

WGQGTLVTVSS (SEQ ID NO:140) or a polypeptide with at least 80%, 85%, 90%, 92%, 94%, 95%, 96%, 97%, 98%, or 99% amino acid sequence identity thereto.

EIVMTQSPATLSLSPGERATLSCRAS (SEQ ID NO:141) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:142) or a polypeptide with at least 80%, 85%, 90%, 92%, 94%, 95%, 96%, 97%, 98%, or 99% amino acid sequence identity thereto.

NRATGIPARFSGSGSGTDYTLTISSLEPEDFAVYYC (SEQ ID NO:143) or a polypeptide with at least 80%, 85%, 90%, 92%, 94%, 95%, 96%, 97%, 98%, or 99% amino acid sequence identity thereto.

FGGGTKLEIK (SEQ ID NO:144) or a polypeptide with at least 80%, 85%, 90%, 92%, 94%, 95%, 96%, 97%, 98%, or 99% amino acid sequence identity thereto.

EVQLVQSGAEVKKPGASVKVSCKAS (SEQ ID NO:145) or a polypeptide with at least 80%, 85%, 90%, 92%, 94%, 95%, 96%, 97%, 98%, or 99% amino acid sequence identity thereto.

MHWVRQAPGQGLEWIGR (SEQ ID NO:146) or a polypeptide with at least 80%, 85%, 90%, 92%, 94%, 95%, 96%, 97%, 98%, or 99% amino acid sequence identity thereto.

KYAPKFQGRVTITADTSTNTAYMELSSLRSEDTAVYYC (SEQ ID NO:147) or a polypeptide with at least 80%, 85%, 90%, 92%, 94%, 95%, 96%, 97%, 98%, or 99% amino acid sequence identity thereto.

WGQGTLVTVSS (SEQ ID NO:148) or a polypeptide with at least 80%, 85%, 90%, 92%, 94%, 95%, 96%, 97%, 98%, or 99% amino acid sequence identity thereto.

DIVMTQSPATLSLSPGERATLSCRAS (SEQ ID NO:149) or a polypeptide with at least 80%, 85%, 90%, 92%, 94%, 95%, 96%, 97%, 98%, or 99% amino acid sequence identity thereto.

LAWYQQKPGQAPRLLVY (SEQ ID NO:150) or a polypeptide with at least 80%, 85%, 90%, 92%, 94%, 95%, 96%, 97%, 98%, or 99% amino acid sequence identity thereto.

NLADGVPARFSGSGSGTDYTLTISSLEPEDFAVYYC (SEQ ID NO:151) or a polypeptide with at least 80%, 85%, 90%, 92%, 94%, 95%, 96%, 97%, 98%, or 99% amino acid sequence identity thereto.

FGGGTKLEIK (SEQ ID NO:152) or a polypeptide with at least 80%, 85%, 90%, 92%, 94%, 95%, 96%, 97%, 98%, or 99% amino acid sequence identity thereto.

EVQLVQSGAEVKKPGASVKVSCKAS (SEQ ID NO:153) or a polypeptide with at least 80%, 85%, 90%, 92%, 94%, 95%, 96%, 97%, 98%, or 99% amino acid sequence identity thereto.

MHWVRQAPGQGLEWIGRI (SEQ ID NO:154) or a polypeptide with at least 80%, 85%, 90%, 92%, 94%, 95%, 96%, 97%, 98%, or 99% amino acid sequence identity thereto.

KYAPKFQGRVTITADTSTNTAYMELSSLRSEDTAVYYC (SEQ ID NO:96) or a polypeptide with at least 80%, 85%, 90%, 92%, 94%, 95%, 96%, 97%, 98%, or 99% amino acid sequence identity thereto.

WGQGTLVTVSS (SEQ ID NO:155) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:156) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:157) or a polypeptide with at least 80%, 85%, 90%, 92%, 94%, 95%, 96%, 97%, 98%, or 99% amino acid sequence identity thereto.

NLASGVPSRFSGSGSGTDYTLTISSLQPEDFATYYC (SEQ ID NO:158) or a polypeptide with at least 80%, 85%, 90%, 92%, 94%, 95%, 96%, 97%, 98%, or 99% amino acid sequence identity thereto.

FGGGTKLEIK (SEQ ID NO:159) or a polypeptide with at least 80%, 85%, 90%, 92%, 94%, 95%, 96%, 97%, 98%, or 99% amino acid sequence identity thereto.

EVQLVQSGAEVKKPGASVKVSCKAS (SEQ ID NO:160) or a polypeptide with at least 80%, 85%, 90%, 92%, 94%, 95%, 96%, 97%, 98%, or 99% amino acid sequence identity thereto.

MHWVRQAPGQGLEWIGR (SEQ ID NO:161) or a polypeptide with at least 80%, 85%, 90%, 92%, 94%, 95%, 96%, 97%, 98%, or 99% amino acid sequence identity thereto.

KYAPKFQGRVTITADTSTNTAYMELSSLRSEDTAVYYC (SEQ ID NO:162) or a polypeptide with at least 80%, 85%, 90%, 92%, 94%, 95%, 96%, 97%, 98%, or 99% amino acid sequence identity thereto.

WGQGTLVTVSS (SEQ ID NO:163) or a polypeptide with at least 80%, 85%, 90%, 92%, 94%, 95%, 96%, 97%, 98%, or 99% amino acid sequence identity thereto.

DIQMTQSPSSLSASVGDRVTITCRAS (SEQ ID NO:164) or a polypeptide with at least 80%, 85%, 90%, 92%, 94%, 95%, 96%, 97%, 98%, or 99% amino acid sequence identity thereto.

LAWYQQKPGKAPKLLVY (SEQ ID NO:165) or a polypeptide with at least 80%, 85%, 90%, 92%, 94%, 95%, 96%, 97%, 98%, or 99% amino acid sequence identity thereto.

NLADGVPSRFSGSGSGTDYTLTISSLQPEDFATYYC (SEQ ID NO:166) FGGGTKLEIK (SEQ ID NO:167), or a polypeptide with at least 80%, 85%, 90%, 92%, 94%, 95%, 96%, 97%, 98%, or 99% amino acid sequence identity thereto.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising an anti-human A-FABP antibody, or an antigen binding fragment thereof, or a polypeptide, that inhibits human A-FABP activity, wherein the antibody, the antigen binding fragment thereof, or the polypeptide has:
i) a variable region comprising a first complementarity determining region (CDR) comprising GFNIKNTY (SEQ ID NO: 1) operably linked to a second CDR comprising IDPANGNT (SEQ ID NO:2) operably linked to a third CDR comprising VSLTGVFAY (SEQ II) NO:3), and/or
ii) a variable region comprising a first CDR comprising ENIYSN (SEQ ID NO:4) operably linked to a second CDR comprising AAT operably linked to a third CDR comprising QHEWGTPWT (SEQ ID NO:6).

2. The composition of claim 2 wherein the antibody fragment is a scFv.

3. The composition of claim 1 which comprises the antibody.

4. The composition of claim 1, wherein the antibody comprises an IgG heavy chain constant region.

5. The composition of claim 1, wherein the antibody comprises an Ig kappa light chain constant region.

6. The composition of claim 1, wherein the antibody comprises an Ig lambda light chain constant region.

7. The composition of claim 1 further comprising a pharmaceutically acceptable carrier.

8. The composition of claim 1, wherein the antibody, fragment thereof, or polypeptide comprises one of SEQ ID NOs: 7 to 38 or a sequence with at least 80% amino acid sequence identity to one of SEQ ID NOS: 40-177.

9. The composition of claim 1, wherein the antibody, fragment thereof, or polypeptide comprises a region having at least 80% amino acid sequence identity to the non-CDR regions in one of SEQ ID NOs: 7 to 38.

10. An isolated cell comprising an expression cassette comprising a heterologous promoter operably linked to nucleic acid sequences encoding a humanized anti-human A-FABP antibody, or an antigen binding fragment thereof, or a polypeptide, that inhibits human A-FABP activity, wherein the antibody, the antigen binding fragment thereof, or the polypeptide has:
i) a variable region comprising a first complementarity determining region (CDR) comprising (SEQ ID NO: 1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6).

11. The cell of claim 10 which is a mammalian cell.

12. The cell of claim 11 wherein the cell is a primate cell.

13. The cell of claim 12 wherein the cell is a human cell.

14. An isolated nucleic acid comprising a promoter operably linked to a nucleotide sequence which encodes at least the variable region of a heavy or light chain that binds human A-FABP, wherein the chain comprises:
i) a variable region comprising a first complementarity determining region (CDR) comprising GFNIKNTY (SEQ ID NO: 1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6).

15. A method to inhibit or treat obesity-associated cancers in a mammal, comprising: administering to a mammal a composition comprising an effective amount of an antibody or fragment thereof comprising, a polypeptide comprising or nucleotide sequence which encodes, at least the variable region of a heavy or light chain that binds human A-FABP, wherein the chain comprises:
i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6), or
an antibody or fragment comprising
i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6).

16. The method of claim 15 wherein the heavy chain is an IgG heavy chain.

17. The method of claim 15, wherein the light chain is an Igκ light chain.

18. The method of claim 15 wherein the antibody fragment is administered.

19. The method of claim 18 wherein the fragment is Fab′ or scFv.

20. A method to diagnose the risk of obesity-associated cancers in a mammal, comprising:
a) contacting a physiological sample from the mammal and at least the variable region of a heavy or light chain that binds human A-FABP, wherein the chain comprises:
i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6), or
an antibody or fragment comprising
i) a variable region comprising a first complementarity determining region (CDR) comprising GFNIKNTY (SEQ ID NO: 1) operably linked to a second CDR comprising IDPANGNT (SEQ ID NO:2) operably linked to a third CDR comprising VSLTGVFAY (SEQ ID NO:3)) and/or
ii) a variable region comprising a first CDR comprising ENIYSN (SEQ ID NO:4) operably linked to a second CDR comprising AAT operably linked to a third CDR comprising QHFWGTPWT (SEQ ID NO:6); and
b) determining the presence or amount of A-FABP in the sample.

21. The method of claim 20 wherein the sample is a physiological fluid sample.

22. The method of claim 21 wherein the sample is a blood, plasma or serum sample.

23. The method of claim 20 wherein the sample is a breast aspirate fluid sample.

24. The method of claim 20, wherein the mammal is human.

25. A method to prevent, inhibit or treat type 2 diabetes, atherosclerosis or heart disease in a mammal, comprising: administering to a mammal a composition comprising an effective amount of an antibody or fragment thereof comprising, a polypeptide comprising or nucleotide sequence which encodes, at least the variable region of a heavy or light chain that binds human A-FABP, wherein the chain comprises:
i) a variable region comprising a first complementarity determining region (CDR) comprising GFNIKNTY (SEQ ID NO: 1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6), or
an antibody or fragment comprising
i) a variable region comprising a first complementarity determining region (CDR) comprising GFNIKNTY (SEQ ID NO:1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6).

26. The method of claim 25 wherein the heavy chain is an IgG heavy chain.

27. The method of claim 25, wherein the light chain is an Igκ light chain.

28. The method of claim 25 wherein the antibody fragment is administered.

29. The method of claim 28 wherein the fragment is Fab′ or scFv.

30. The method of claim 25, wherein the mammal is human.

31. The method of claim 25, wherein the mammal has heart disease.

32. The method of claim 25, wherein the mammal has type 2 diabetes.

33. The method of claim 25, wherein the mammal is obese.

34. The method of claim 15, wherein the composition is systemically administered.

35. The method of claim 15, wherein the composition is injected.

36. A method to diagnose the risk of type 2 diabetes, atherosclerosis or heart disease in a mammal, comprising:
a) contacting a physiological sample from the mammal and at least the variable region of a heavy or light chain that binds human A-FABP, wherein the chain comprises:
i) a variable region comprising a first complementarity determining region (CDR) comprising GFNIKNTY (SEQ ID NO: 1) operably linked to a second CDR comprising IDPANGNT (SEQ ID NO:2) operably linked to a third CDR comprising VSLTGVFAY (SEQ ID NO:3); and/or
ii) a variable region comprising a first CDR comprising ENIYSN (SEQ ID NO:4) operably linked to a second CDR comprising AAT operably linked to a third CDR comprising QHFWGTPWT (SEQ ID NO:6), or
an antibody or fragment comprising
i) a variable region comprising a first complementarity determining region (CDR) comprising GFNIKNTY (SEQ ID NO: 1) operably linked to a second CDR comprising IDPANGNT (SEQ ID NO:2) operably linked to a third CDR comprising VSLTGVFAY (SEQ ID NO:3), and/or
ii) a variable region comprising a first CDR comprising ENIYSN (SEQ ID NO:4) operably linked to a second CDR comprising AAT operably linked to a third CDR comprising QHFWGTPWT (SEQ ID NO:6); and
b) determining the presence or amount of A-FABP in the sample.

37. The method of claim 36 wherein the sample is a physiological fluid sample.

38. The method of claim 37 wherein the sample is a blood, serum or plasma sample.

39. The method of claim 37, wherein the mammal is human.


SEQUENCE LISTING (210 sequences)

SEQ ID NO: 1 - protein, 8 aa, synthetic construct
GFNIKNTY

SEQ ID NO: 2 - protein, 8 aa, synthetic construct
IDPANGNT

SEQ ID NO: 3 - protein, 9 aa, synthetic construct
VSLTGVFAY

SEQ ID NO: 4 - protein, 6 aa, synthetic construct
ENIYSN

SEQ ID NO: 5 - skipped

SEQ ID NO: 6 - protein, 9 aa, synthetic construct
QHFWGTPWT

SEQ ID NO: 7 - protein, 116 aa, synthetic construct
EVQLVQSGAEVKKPGESLKISCKASGFNIKNTYIGWVRQMPGKGLEWMGRIDPANGNTKYAPSFQGQVTISADTSISTAYLQWSSLKASDTAMYYCVSLTGVFAYWGQGTLVTVSS

SEQ ID NO: 8 - protein, 107 aa, synthetic construct
EIVMTQSPATLSLSPGERATLSCRASENIYSNLAWYQQKPGQAPRLLVYAATNRATGIPARFSGSGSGTDYTLTISSLEPEDFAVYYCQHFWGTPWTFGGGTKLEIK

SEQ ID NO: 9 - protein, 116 aa, synthetic construct; identical to SEQ ID NO: 7

SEQ ID NO: 10 - protein, 107 aa, synthetic construct
DIVMTQSPATLSLSPGERATLSCRASENIYSNLAWYQQKPGQAPRLLVYAATNLADGVPARFSGSGSGTDYTLTISSLEPEDFAVYYCQHFWGTPWTFGGGTKLEIK

SEQ ID NO: 11 - protein, 116 aa, synthetic construct; identical to SEQ ID NO: 7

SEQ ID NO: 12 - protein, 107 aa, synthetic construct
DIQMTQSPSSLSASVGDRVTITCRASENIYSNLAWYQQKPGKAPKLLVYAATNLASGVPSRFSGSGSGTDYTLTISSLQPEDFATYYCQHFWGTPWTFGGGTKLEIK

SEQ ID NO: 13 - protein, 116 aa, synthetic construct; identical to SEQ ID NO: 7

SEQ ID NO: 14 - protein, 107 aa, synthetic construct
DIQMTQSPSSLSASVGDRVTITCRASENIYSNLAWYQQKPGKAPKLLVYAATNLADGVPSRFSGSGSGTDYTLTISSLQPEDFATYYCQHFWGTPWTFGGGTKLEIK

SEQ ID NO: 15 - protein, 116 aa, synthetic construct
EVQLVQSGAEVKKPGESLKISCKASGFNIKNTYMHWVRQMPGKGLEWIGRIDPANGNTKYAPSFQGQVTISADTSINTAYLQWSSLKASDTAMYYCVSLTGVFAYWGQGTLVTVSS

SEQ ID NO: 16 - protein, 107 aa, synthetic construct; identical to SEQ ID NO: 8

SEQ ID NO: 17 - protein, 116 aa, synthetic construct; identical to SEQ ID NO: 15

SEQ ID NO: 18 - protein, 107 aa, synthetic construct; identical to SEQ ID NO: 10

SEQ ID NO: 19 - protein, 116 aa, synthetic construct; identical to SEQ ID NO: 15

SEQ ID NO: 20 - protein, 107 aa, synthetic construct; identical to SEQ ID NO: 12

SEQ ID NO: 21 - protein, 116 aa, synthetic construct; identical to SEQ ID NO: 15

SEQ ID NO: 22 - protein, 107 aa, synthetic construct; identical to SEQ ID NO: 14

SEQ ID NO: 23 - protein, 116 aa, synthetic construct
QVQLVQSGAEVKKPGASVKVSCKASGFNIKNTYMHWVRQAPGQGLEWMGRIDPANGNTKYAPKFQGRVTMTADTSTSTAYMELSSLRSEDTAVYYCVSLTGVFAYWGQGTLVTVSS

SEQ ID NO: 24 - protein, 107 aa, synthetic construct; identical to SEQ ID NO: 8

SEQ ID NO: 25 - protein, 115 aa, synthetic construct
VQLVQSGAEVKKPGASVKVSCKASGFNIKNTYMHWVRQAPGQGLEWMGRIDPANGNTKYAPKFQGRVTMTADTSTSTAYMELSSLRSEDTAVYYCVSLTGVFAYWGQGTLVTVSS

SEQ ID NO: 26 - protein, 107 aa, synthetic construct; identical to SEQ ID NO: 10

SEQ ID NO: 27 - protein, 116 aa, synthetic construct; identical to SEQ ID NO: 23

SEQ ID NO: 28 - protein, 107 aa, synthetic construct; identical to SEQ ID NO: 12

SEQ ID NO: 29 - protein, 116 aa, synthetic construct; identical to SEQ ID NO: 23

SEQ ID NO: 30 - protein, 107 aa, synthetic construct; identical to SEQ ID NO: 14

SEQ ID NO: 31 - protein, 116 aa, synthetic construct
EVQLVQSGAEVKKPGASVKVSCKASGFNIKNTYMHWVRQAPGQGLEWIGRIDPANGNTKYAPKFQGRVTITADTSTNTAYMELSSLRSEDTAVYYCVSLTGVFAYWGQGTLVTVSS

SEQ ID NO: 32 - protein, 107 aa, synthetic construct; identical to SEQ ID NO: 8

SEQ ID NO: 33 - protein, 116 aa, synthetic construct; identical to SEQ ID NO: 31

SEQ ID NO: 34 - protein, 107 aa, synthetic construct; identical to SEQ ID NO: 10

SEQ ID NO: 35 - protein, 116 aa, synthetic construct; identical to SEQ ID NO: 31

SEQ ID NO: 36 - protein, 107 aa, synthetic construct; identical to SEQ ID NO: 12

SEQ ID NO: 37 - protein, 116 aa, synthetic construct; identical to SEQ ID NO: 31

SEQ ID NO: 38 - protein, 107 aa, synthetic construct; identical to SEQ ID NO: 14

SEQ ID NO: 39 - skipped

SEQ ID NO: 40 - protein, 25 aa, synthetic construct
EVQLVQSGAEVKKPGESLKISCKAS

SEQ ID NO: 41 - protein, 17 aa, synthetic construct
IGWVRQMPGKGLEWMGR

SEQ ID NO: 42 - protein, 38 aa, synthetic construct
KYAPSFQGQVTISADTSISTAYLQWSSLKASDTAMYYC

SEQ ID NO: 43 - protein, 12 aa, synthetic construct
YWGQGTLVTVSS

SEQ ID NO: 44 - protein, 26 aa, synthetic construct
EIVMTQSPATLSLSPGERATLSCRAS

SEQ ID NO: 45 - protein, 17 aa, synthetic construct
LAWYQQKPGQAPRLLVY

SEQ ID NO: 46 - protein, 36 aa, synthetic construct
NRATGIPARFSGSGSGTDYTLTISSLEPEDFAVYYC

SEQ ID NO: 47 - protein, 10 aa, synthetic construct
FGGGTKLEIK

SEQ ID NO: 48 - protein, 25 aa, synthetic construct
EVQLVQSGAEVKKPGESLKISCKAS

SEQ ID NO: 49 - protein, 17 aa, synthetic construct
IGWVRQMPGKGLEWMGR

SEQ ID NO: 50 - protein, 38 aa, synthetic construct; identical to SEQ ID NO: 42

SEQ ID NO: 51 - protein, 11 aa, synthetic construct
WGQGTLVTVSS

SEQ ID NO: 52 - protein, 26 aa, synthetic construct
DIVMTQSPATLSLSPGERATLSCRAS

SEQ ID NO: 53 - protein, 17 aa, synthetic construct
LAWYQQKPGQAPRLLVY

SEQ ID NO: 54 - protein, 36 aa, synthetic construct
NLADGVPARFSGSGSGTDYTLTISSLEPEDFAVYYC

SEQ ID NO: 55 - protein, 10 aa, synthetic construct
FGGGTKLEIK

SEQ ID NO: 56 - protein, 25 aa, synthetic construct
EVQLVQSGAEVKKPGESLKISCKAS

SEQ ID NO: 57 - protein, 17 aa, synthetic construct
IGWVRQMPGKGLEWMGR

SEQ ID NO: 58 - protein, 38 aa, synthetic construct; identical to SEQ ID NO: 42

SEQ ID NO: 59 - protein, 11 aa, synthetic construct
WGQGTLVTVSS

SEQ ID NO: 60 - protein, 26 aa, synthetic construct [sequence omitted]

SEQ ID NO: 61 - protein, 17 aa, synthetic construct
LAWYQQKPGKAPKLLVY

SEQ ID NO: 62 - protein, 36 aa, synthetic construct
NLASGVPSRFSGSGSGTDYTLTISSLQPEDFATYYC

SEQ ID NO: 63 - protein, 10 aa, synthetic construct
FGGGTKLEIK

SEQ ID NO: 64 - protein, 25 aa, synthetic construct
EVQLVQSGAEVKKPGESLKISCKAS

SEQ ID NO: 65 - protein, 17 aa, synthetic construct
IGWVRQMPGKGLEWMGR

SEQ ID NO: 66 - protein, 38 aa, synthetic construct; identical to SEQ ID NO: 42

SEQ ID NO: 67 - protein, 11 aa, synthetic construct
WGQGTLVTVSS

SEQ ID NO: 68 - protein, 26 aa, synthetic construct [sequence omitted]

SEQ ID NO: 69 - protein, 17 aa, synthetic construct
LAWYQQKPGKAPKLLVY

SEQ ID NO: 70 - protein, 36 aa, synthetic construct
NLADGVPSRFSGSGSGTDYTLTISSLQPEDFATYYC

SEQ ID NO: 71 - protein, 10 aa, synthetic construct
FGGGTKLEIK

SEQ ID NO: 72 - protein, 25 aa, synthetic construct
EVQLVQSGAEVKKPGESLKISCKAS

SEQ ID NO: 73 - protein, 17 aa, synthetic construct
MHWVRQMPGKGLEWIGR

SEQ ID NO: 74 - protein, 38 aa, synthetic construct
KYAPSFQGQVTISADTSINTAYLQWSSLKASDTAMYYC

SEQ ID NO: 75 - protein, 11 aa, synthetic construct
WGQGTLVTVSS

SEQ ID NO: 76 - protein, 26 aa, synthetic construct
EIVMTQSPATLSLSPGERATLSCRAS

SEQ ID NO: 77 - protein, 17 aa, synthetic construct
LAWYQQKPGQAPRLLVY

SEQ ID NO: 78 - protein, 36 aa, synthetic construct; identical to SEQ ID NO: 46

SEQ ID NO: 79 - protein, 10 aa, synthetic construct
FGGGTKLEIK

SEQ ID NO: 80 - protein, 25 aa, synthetic construct
EVQLVQSGAEVKKPGESLKISCKAS

SEQ ID NO: 81 - protein, 17 aa, synthetic construct
MHWVRQMPGKGLEWIGR

SEQ ID NO: 82 - protein, 38 aa, synthetic construct; identical to SEQ ID NO: 74

SEQ ID NO: 83 - protein, 11 aa, synthetic construct
WGQGTLVTVSS

SEQ ID NO: 84 - protein, 26 aa, synthetic construct
DIVMTQSPATLSLSPGERATLSCRAS

SEQ ID NO: 85 - protein, 17 aa, synthetic construct
LAWYQQKPGQAPRLLVY

SEQ ID NO: 86 - protein, 36 aa, synthetic construct; identical to SEQ ID NO: 54

SEQ ID NO: 87 - protein, 10 aa, synthetic construct
FGGGTKLEIK

SEQ ID NO: 88 - protein, 25 aa, synthetic construct
EVQLVQSGAEVKKPGESLKISCKAS

SEQ ID NO: 89 - protein, 17 aa, synthetic construct
MHWVRQMPGKGLEWIGR

SEQ ID NO: 90 - protein, 38 aa, synthetic construct; identical to SEQ ID NO: 74

SEQ ID NO: 91 - protein, 11 aa, synthetic construct
WGQGTLVTVSS

SEQ ID NO: 92 - protein, 26 aa, synthetic construct [sequence omitted]

SEQ ID NO: 93 - protein, 17 aa, synthetic construct
LAWYQQKPGKAPKLLVY

SEQ ID NO: 94 - protein, 36 aa, synthetic construct; identical to SEQ ID NO: 62

SEQ ID NO: 95 - protein, 10 aa, synthetic construct
FGGGTKLEIK

SEQ ID NO: 96 - protein, 38 aa, synthetic construct
KYAPKFQGRVTITADTSTNTAYMELSSLRSEDTAVYYC

SEQ ID NO: 97 - protein, 25 aa, synthetic construct
EVQLVQSGAEVKKPGESLKISCKAS

SEQ ID NO: 98 - protein, 17 aa, synthetic construct
MHWVRQMPGKGLEWIGR

SEQ ID NO: 99 - protein, 38 aa, synthetic construct; identical to SEQ ID NO: 74

SEQ ID NO: 100 - protein, 11 aa, synthetic construct
WGQGTLVTVSS

SEQ ID NO: 101 - protein, 26 aa, synthetic construct [sequence omitted]

SEQ ID NO: 102 - protein, 17 aa, synthetic construct
LAWYQQKPGKAPKLLVY

SEQ ID NO: 103 - protein, 36 aa, synthetic construct; identical to SEQ ID NO: 70

SEQ ID NO: 104 - protein, 10 aa, synthetic construct
FGGGTKLEIK

SEQ ID NO: 105 - protein, 25 aa, synthetic construct [sequence omitted]

SEQ ID NO: 106 - protein, 17 aa, synthetic construct
MHWVRQAPGQGLEWMGR

SEQ ID NO: 107 - protein, 38 aa, synthetic construct
KYAPKFQGRVTMTADTSTSTAYMELSSLRSEDTAVYYC

SEQ ID NO: 108 - protein, 11 aa, synthetic construct
WGQGTLVTVSS

SEQ ID NO: 109 - protein, 26 aa, synthetic construct
EIVMTQSPATLSLSPGERATLSCRAS

SEQ ID NO: 110 - protein, 17 aa, synthetic construct
LAWYQQKPGQAPRLLVY

SEQ ID NO: 111 - protein, 36 aa, synthetic construct; identical to SEQ ID NO: 46

SEQ ID NO: 112 - protein, 10 aa, synthetic construct
FGGGTKLEIK

SEQ ID NO: 113 - protein, 24 aa, synthetic construct
VQLVQSGAEVKKPGASVKVSCKAS

SEQ ID NO: 114 - protein, 17 aa, synthetic construct
MHWVRQAPGQGLEWMGR

SEQ ID NO: 115 - protein, 38 aa, synthetic construct; identical to SEQ ID NO: 107

SEQ ID NO: 116 - protein, 11 aa, synthetic construct
WGQGTLVTVSS

SEQ ID NO: 117 - protein, 26 aa, synthetic construct
DIVMTQSPATLSLSPGERATLSCRAS

SEQ ID NO: 118 - protein, 17 aa, synthetic construct
LAWYQQKPGQAPRLLVY

SEQ ID NO: 119 - protein, 36 aa, synthetic construct; identical to SEQ ID NO: 54

SEQ ID NO: 120 - protein, 10 aa, synthetic construct
FGGGTKLEIK

SEQ ID NO: 121 - protein, 25 aa, synthetic construct [sequence omitted]

SEQ ID NO: 122 - protein, 17 aa, synthetic construct
MHWVRQAPGQGLEWMGR

SEQ ID NO: 123 - protein, 38 aa, synthetic construct; identical to SEQ ID NO: 107

SEQ ID NO: 124 - protein, 11 aa, synthetic construct
WGQGTLVTVSS

SEQ ID NO: 125 - protein, 26 aa, synthetic construct [sequence omitted]

SEQ ID NO: 126 - protein, 17 aa, synthetic construct
LAWYQQKPGKAPKLLVY

SEQ ID NO: 127 - protein, 37 aa, synthetic construct
NLASGVPSRFSGSGSGTDYTLTISSLQPEDFATYYCQ

SEQ ID NO: 128 - protein, 9 aa, synthetic construct
GGGTKLEIK

SEQ ID NO: 129 - protein, 25 aa, synthetic construct [sequence omitted]

SEQ ID NO: 130 - protein, 17 aa, synthetic construct
MHWVRQAPGQGLEWMGR

SEQ ID NO: 131 - protein, 38 aa, synthetic construct; identical to SEQ ID NO: 107

SEQ ID NO: 132 - protein, 11 aa, synthetic construct
WGQGTLVTVSS

SEQ ID NO: 133 - protein, 26 aa, synthetic construct [sequence omitted]

SEQ ID NO: 134 - protein, 17 aa, synthetic construct
LAWYQQKPGKAPKLLVY

SEQ ID NO: 135 - protein, 36 aa, synthetic construct; identical to SEQ ID NO: 70

SEQ ID NO: 136 - protein, 10 aa, synthetic construct
FGGGTKLEIK

SEQ ID NO: 137 - protein, 25 aa, synthetic construct
EVQLVQSGAEVKKPGASVKVSCKAS

SEQ ID NO: 138 - protein, 17 aa, synthetic construct
MHWVRQAPGQGLEWIGR

SEQ ID NO: 139 - protein, 38 aa, synthetic construct; identical to SEQ ID NO: 96

SEQ ID NO: 140 - protein, 11 aa, synthetic construct
WGQGTLVTVSS

SEQ ID NO: 141 - protein, 26 aa, synthetic construct
EIVMTQSPATLSLSPGERATLSCRAS

SEQ ID NO: 142 - protein, 17 aa, synthetic construct
LAWYQQKPGQAPRLLVY

SEQ ID NO: 143 - protein, 36 aa, synthetic construct; identical to SEQ ID NO: 46

SEQ ID NO: 144 - protein, 10 aa, synthetic construct
FGGGTKLEIK

SEQ ID NO: 145 - protein, 25 aa, synthetic construct
EVQLVQSGAEVKKPGASVKVSCKAS

SEQ ID NO: 146 - protein, 17 aa, synthetic construct
MHWVRQAPGQGLEWIGR

SEQ ID NO: 147 - protein, 38 aa, synthetic construct; identical to SEQ ID NO: 96

SEQ ID NO: 148 - protein, 11 aa, synthetic construct
WGQGTLVTVSS

SEQ ID NO: 149 - protein, 26 aa, synthetic construct
DIVMTQSPATLSLSPGERATLSCRAS

SEQ ID NO: 150 - protein, 17 aa, synthetic construct
LAWYQQKPGQAPRLLVY

SEQ ID NO: 151 - protein, 36 aa, synthetic construct; identical to SEQ ID NO: 54

SEQ ID NO: 152 - protein, 10 aa, synthetic construct
FGGGTKLEIK

SEQ ID NO: 153 - protein, 25 aa, synthetic construct
EVQLVQSGAEVKKPGASVKVSCKAS

SEQ ID NO: 154 - protein, 18 aa, synthetic construct
MHWVRQAPGQGLEWIGRI

SEQ ID NO: 155 - protein, 11 aa, synthetic construct
WGQGTLVTVSS

SEQ ID NO: 156 - protein, 26 aa, synthetic construct [sequence omitted]

SEQ ID NO: 157 - protein, 17 aa, synthetic construct
LAWYQQKPGKAPKLLVY

SEQ ID NO: 158 - protein, 36 aa, synthetic construct; identical to SEQ ID NO: 62

SEQ ID NO: 159 - protein, 10 aa, synthetic construct
FGGGTKLEIK

SEQ ID NO: 160 - protein, 25 aa, synthetic construct
EVQLVQSGAEVKKPGASVKVSCKAS

SEQ ID NO: 161 - protein, 17 aa, synthetic construct
MHWVRQAPGQGLEWIGR

SEQ ID NO: 162 - protein, 38 aa, synthetic construct; identical to SEQ ID NO: 96

SEQ ID NO: 163 - protein, 11 aa, synthetic construct
WGQGTLVTVSS

SEQ ID NO: 164 - protein, 26 aa, synthetic construct [sequence omitted]

SEQ ID NO: 165 - protein, 17 aa, synthetic construct
LAWYQQKPGKAPKLLVY

SEQ ID NO: 166 - protein, 36 aa, synthetic construct; identical to SEQ ID NO: 70

SEQ ID NO: 167 - protein, 10 aa, synthetic construct
FGGGTKLEIK

SEQ ID NO: 168 - protein, 116 aa, synthetic construct
EVQLQQSVAELVRPGASVKLSCTASGFNIKNTYMHWVKQRPEQGLEWIGRIDPANGNTKYAPKFQGKATITADTSSNTAYLQLSSLTSEDTAIYYCVSLTGVFAYWGQGTLVTVSA

SEQ ID NO: 169 - protein, 107 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 170 - protein, 116 aa, synthetic construct; identical to SEQ ID NO: 31

SEQ ID NO: 171 - protein, 116 aa, synthetic construct; identical to SEQ ID NO: 23

SEQ ID NO: 172 - protein, 116 aa, synthetic construct; identical to SEQ ID NO: 15

SEQ ID NO: 173 - protein, 116 aa, synthetic construct; identical to SEQ ID NO: 7

SEQ ID NO: 174 - protein, 107 aa, synthetic construct; identical to SEQ ID NO: 14

SEQ ID NO: 175 - protein, 107 aa, synthetic construct; identical to SEQ ID NO: 12

SEQ ID NO: 176 - protein, 107 aa, synthetic construct; identical to SEQ ID NO: 10

SEQ ID NO: 177 - protein, 107 aa, synthetic construct; identical to SEQ ID NO: 8

SEQ ID NO: 178 - protein, 132 aa, synthetic construct
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 179 - protein, 116 aa, synthetic construct; identical to SEQ ID NO: 7

SEQ ID NO: 180 - protein, 116 aa, synthetic construct; identical to SEQ ID NO: 7

SEQ ID NO: 181 - protein, 116 aa, synthetic construct; identical to SEQ ID NO: 7

SEQ ID NO: 182 - protein, 116 aa, synthetic construct; identical to SEQ ID NO: 7

SEQ ID NO: 183 - protein, 116 aa, synthetic construct; identical to SEQ ID NO: 15

SEQ ID NO: 184 - protein, 116 aa, synthetic construct; identical to SEQ ID NO: 15

SEQ ID NO: 185 - protein, 116 aa, synthetic construct; identical to SEQ ID NO: 15

SEQ ID NO: 186 - protein, 116 aa, synthetic construct; identical to SEQ ID NO: 15

SEQ ID NO: 187 - protein, 116 aa, synthetic construct; identical to SEQ ID NO: 23

SEQ ID NO: 188 - protein, 116 aa, synthetic construct; identical to SEQ ID NO: 23

SEQ ID NO: 189 - protein, 116 aa, synthetic construct; identical to SEQ ID NO: 23

SEQ ID NO: 190 - protein, 116 aa, synthetic construct; identical to SEQ ID NO: 23

SEQ ID NO: 191 - protein, 116 aa, synthetic construct; identical to SEQ ID NO: 31

SEQ ID NO: 192 - protein, 116 aa, synthetic construct; identical to SEQ ID NO: 31

SEQ ID NO: 193 - protein, 116 aa, synthetic construct; identical to SEQ ID NO: 31

SEQ ID NO: 194 - protein, 116 aa, synthetic construct; identical to SEQ ID NO: 31

SEQ ID NO: 195 - protein, 107 aa, synthetic construct; identical to SEQ ID NO: 8

SEQ ID NO: 196 - protein, 107 aa, synthetic construct; identical to SEQ ID NO: 10

SEQ ID NO: 197 - protein, 107 aa, unidentified; identical to SEQ ID NO: 12

SEQ ID NO: 198 - protein, 107 aa, synthetic construct; identical to SEQ ID NO: 14

SEQ ID NO: 199 - protein, 107 aa, synthetic construct; identical to SEQ ID NO: 8

SEQ ID NO: 200 - protein, 108 aa, synthetic construct
DIVMTQSPATLSLSPTGERATLSCRASENIYSNLAWYQQKPGQAPRLLVYAATNLADGVPARFSGSGSGTDYTLTISSLEPEDFAVYYCQHFWGTPWTFGGGTKLEIK

SEQ ID NO: 201 - protein, 107 aa, synthetic construct; identical to SEQ ID NO: 12

SEQ ID NO: 202 - protein, 108 aa, synthetic construct
DIQMTQSPSSLSASVGDRVTITCRASENIYSNLAWYQQKPGKAPKLLVYAATNLADGVPSRFSGSGSGTYDYTLTISSLQPEDFATYYCQHFWGTPWTFGGGTKLEIK

SEQ ID NO: 203 - protein, 107 aa, synthetic construct; identical to SEQ ID NO: 8

SEQ ID NO: 204 - protein, 107 aa, synthetic construct; identical to SEQ ID NO: 10

SEQ ID NO: 205 - protein, 107 aa, synthetic construct; identical to SEQ ID NO: 12

SEQ ID NO: 206 - protein, 107 aa, synthetic construct; identical to SEQ ID NO: 14

SEQ ID NO: 207 - protein, 107 aa, synthetic construct; identical to SEQ ID NO: 8

SEQ ID NO: 208 - protein, 107 aa, synthetic construct; identical to SEQ ID NO: 10

SEQ ID NO: 209 - protein, 107 aa, synthetic construct; identical to SEQ ID NO: 12

SEQ ID NO: 210 - protein, 107 aa, synthetic construct; identical to SEQ ID NO: 14